(19) United States
(12) Patent Application Publication
(10) Pub. No.: US 2023/0064414 A1
(43) Pub. Date: Mar. 2, 2023

(54) SYSTEM AND METHOD FOR AUTOMATED AND SEMI-AUTOMATED MOSQUITO SEPARATION IDENTIFICATION COUNTING AND POOLING

(71) Applicant: Senecio Ltd., Kfar-Saba (IL)

(72) Inventors: Hanan LEPEK, Kfar-Saba (IL); Yeshayahu SHAPIRA, Kfar Saba (IL); Gabriel WEIZMAN, Shoham (IL)

(73) Assignee: Senecio Ltd., Kfar-Saba (IL)

(21) Appl. No.: 17/777,090

(22) PCT Filed: Nov. 15, 2020
(86) PCT No.: PCT/IL2020/051182

§ 371 Date: May 16, 2022

Related U.S. Application Data
(60) Provisional application No. 63/007,064, filed on Apr. 8, 2020.
(60) Provisional application No. 62/988,427, filed on Mar. 12, 2020.
(60) Provisional application No. 62/935,414, filed on Nov. 14, 2019.

(51) Int. Cl. A01M 1/02; G06T 7/70; G06V 20/50; G06V 10/764

(52) U.S. Cl. CPC A01M 1/026; G06T 7/70; G06V 20/50; G06V 10/764

(57) ABSTRACT

A method of separating a batch of insects from a trap into individual insects comprises pouring the batch into a container having at least one hole, the hole being sized for a single insect; and moving the container to shake the insects within so that individual insects are caused to exit via the hole onto a collecting surface, thereby providing separated insects onto the collecting surface. The insects from the trap may then be counted and image recognition may be used to identify the genus, species or gender. The process may be carried out for multiple traps and location data may be stored with the insect identifications to give a map of insect distribution.

### RELATED APPLICATION/S

This application claims the benefit of priority under 35 USC § 119(e) of U.S. Provisional Patent Application No. 62/935,414 filed Nov. 14, 2019, U.S. Provisional Application No. 63/007,064 filed Apr. 8, 2020, and 62/988,427 filed Mar. 12, 2020, the contents of which are incorporated herein by reference in their entirety.

### FIELD AND BACKGROUND OF THE INVENTION

The present invention relates to insect surveillance and more particularly but not exclusively to a system and method for surveilling insect populations that may include separation, identification, counting and pooling of the insects found.

In many circumstances it is necessary or desired to monitor insect populations. Such circumstances range from surveying natural ecosystems and monitoring changes, to situations of public health, where insects such as mosquitoes, blackflies, midges and sandflies, present a public health hazard to humans or livestock.

Understanding how many mosquitoes there are in a given area is an important factor for the authorities in order to make decisions regarding control, including the spraying of chemicals, at what quantities, where and so on.

Measuring is today performed by spreading mosquito traps within an area and counting the trapped insects.

When bringing back mosquito traps into the lab, it usually takes a few days until the mosquitoes from that trap are counted and analyzed.

Traps brought to the lab are poured onto a petri dish, roughly cleaned and are then counted.

During the counting process a technician may place a petri dish under the microscope, and then using tweezers take insects one by one and inspect for species and sex as necessary.

Another associated problem with the process to date, is that the employees that are trying to identify correctly the insect species cannot always tell the species of all kinds of insects collected

In addition, usually it is required to randomly pick a certain number of insects of a specific type from the collected insects coming from the traps and store them in small tubes to be sent for laboratories to test if the insects are carrying any viruses.

At times, if there is a SIT (sterile Insect Technique) program in place, it may be also important to count how many are wild, and how many are insects that have been previously intentionally released by personal.

In summary, insect surveillance, and in particular mosquito surveillance requires costly and tedious work, as expert personnel identify, count, and pool mosquitoes one-by-one from hundreds of field traps. The work is exhaustive, and available capacity limits the number of traps surveyed. As a result, there is a large rate of human error, and the overall surveillance is inefficient and not as effective as needed.

### SUMMARY OF THE INVENTION

The present inventions may provide a method. and an apparatus for automated. insect counting, for automated species recognition and pooling, and consequently reducing costs and time while increasing accuracy and consistency.

The apparatus may automatically separate, sort, identify, count and map the processed insects, for example mosquitoes, include for each insect identification an indication of the field. traps that the respective insect came from, and updating real world data as per the mosquito population at the field traps coordinates.

The solution may provide advantages including: Eliminating tedious and repetitive work, saving time, and reducing human errors which may arise in manual processes.

According to one aspect of the present invention there is provided a method of separating a batch of insects from a trap into individual insects comprising:
pouring the batch of insects into a container having at least one hole, the hole being sized for a single insect;
moving the container to shake the insects within so that individual insects are caused to exit via the hole onto a collecting surface, thereby providing separated insects onto the collecting surface.

In an embodiment the container comprises a floor, the motion comprises vibration, and the at least one hole is in the floor.

In an embodiment, the container comprises a circumference, the at least one hole is in the circumference and the motion comprises rotation.

In an embodiment, the motion further comprises vibration.

In an embodiment, the rotation and the vibration are alternated in a cycle.

In an embodiment, the container comprises an upper cone and a lower cone, the cones meeting at a common base, the base providing a maximal circumference and the at least one hole being at the maximal circumference.

Embodiments may involve pouring a batch of insects into the container via a funnel.

In an embodiment, the collecting surface is a moving surface.

According to a second aspect of the present invention there is provided apparatus for separating insects from a batch of insects into individuals, the apparatus comprising a container for the batch of insects, the container being motorized to provide motion to the container, and having at least one hole, the hole sized for an individual insect thereby to enable an individual insect from the batch to be pushed out of the hole when nudged against the hole by the motion.

In an embodiment, the container has a floor, the at least one hole is in the floor and the motion is vibrating motion.

In an embodiment, the container has a circumference, the motion comprises rotation in an axis perpendicular to the circumference and the at least one hole is in the circumference.

In an embodiment, the motion further comprises vibration in at least one axis.

In an embodiment, the motion comprises vibration in three axes.

In an embodiment, the at least one hole comprises an inner side towards an interior of the container and an outer side towards an exterior of the container, and a diameter which is smaller at the inner side than at the outer side.

In an embodiment, the container comprises an upper cone and a lower cone, the cones meeting at a common base, the base providing a maximal circumference and the at least one hole being at the maximal circumference.

An embodiment may have a guide for guiding exiting insects from the at least one hole to a collecting surface.

Embodiments may comprise a funnel for pouring the batch of insects from a trap into the container.

Embodiments may comprise a motor with an eccentric weight to provide vibrations.

According to a third aspect of the present invention there is provided a method of picking an insect on a first surface and placing the insect, the method comprising:

Imaging the collecting surface from above;

From the imaging determining the presence of the insect on the surface for picking;

From the imaging determining a current location of the insect on the surface as a picking location;

Using a robot arm, moving a picking tool to a positon above the picking location;

Lowering the picking tool to the picking location;

Operating suction to pick the insect into the picking tool from the picking location;

Using the robot arm to move the picking tool with the insect to a position above a depositing location; and

Removing the suction to deposit the insect, wherein one of the picking location and the depositing location is an identification location for imaging the insect for identification.

In an embodiment, the identification location is the picking location and an identification made at the identification location defines the depositing location.

The method may comprise switching from the suction to blowing at the depositing location to deposit the insect.

In an embodiment, the picking tool comprises a porous surface in a tube leading to a vacuum source, the insect being held at the porous surface by the suction.

In an embodiment, the picking tool has a central air duct and a peripheral air duct, the suction being applied via the central air duct and the blowing being provided by both the central air duct and the peripheral air duct.

According to a fourth aspect of the present invention there is provided a picking tool for insects comprising a hollow tube having a first end and a second end, the tube being connected to an air pressure source at the first end and having a porous surface proximal to the second end, the tool further having a robot arm for positioning the tool in three dimensions, the tool being configured to work with an imaging system to position itself above coordinates supplied by the imaging system as the position of an insect on a surface, the tool being configured to lower itself over the coordinates and to apply suction to suck the insect against the porous surface thereby to pick the insect.

In an embodiment, the net is distanced from the second end by the thickness of an insect.

The tool may have a central air duct and a peripheral air duct, the suction being applied through the central air duct, thereby to positon the picked insect centrally on the net.

The tool may switch off the suction when reaching a destination, thereby to deposit the insect at a placing location.

Alternatively, the tool may switch off the suction when reaching a destination, and may replace the suction with blowing, the blowing being applied via the central air duct and the peripheral air duct, thereby to deposit the insect at the placing location.

According to a fifth aspect of the present invention there is provided a method of identifying and counting images obtained in batches from field traps, the method comprising:

Receiving a batch of insects from a trap;

Placing the batch into a separator, the separator ejecting insects from the batch one by one;

Collecting the insects being ejected on a moving surface; and

For each the insect on the moving surface taking at least one image; and

For each individual insect found in respective images, incrementing a count.

The method may comprise taking a series of images from different angles for each insect on the moving surface and providing the images to a neural network to identify the insect.

The method may comprise using the identification to define a destination to place the insect.

The method may comprise using a first camera to locate the insect and a second camera to take images from different angles around the insect.

The method may comprise rotating the insect on a rotating disc to obtain the images from different angles.

The method may comprise placing the second camera on a robot arm and moving the second camera around the insect to obtain the images.

The method may comprise illuminating the insect with an excitation wavelength to elicit fluorescence.

The method may comprise obtaining images at different focal depths.

The method may comprise identifying an attitude of the insect and obtaining images of body parts according to locations defined by the attitude.

The method may comprise using a decision tree to define species defining features and positioning the second camera to image body parts according to the decision tree.

The method may comprise operating the separator to eject separated insects onto a length of conveyor, then stopping the separator and identifying insects on the conveyor, and repeating the operating and identifying.

The method may comprise using the identification to define a destination for placing a respective insect.

A plurality of batches may be obtained from a plurality of traps, each trap having a different location, and the method may use insect numbers from respective traps to generate or update a report or a geographical map of insect distribution.

In an embodiment, the identifying is based on an insect database of insects expected in a region of the trap.

In an embodiment, the identifying comprises leaving some insects uncategorized due to being unidentified, or identified to below a threshold level of certainty, the method comprising forwarding images of the uncategorized insects for manual identification by an operator.

According to a further aspect of the present invention there is provided a method of automatically identifying an insect at an imaging location for genus, species or sex, the method comprising:

Taking a first image of the insect from above to identify an orientation of the insects; and

Using the first image to find at least one location from which a first given body part may be imaged, and sending a camera to the location to take a second image; and

Continuing with further locations and further body parts until sufficient images are available to enable identification of the insect.

In an embodiment, the first image is taken using a first camera located overhead and the second image is taken from a second camera on a robot arm.

In an embodiment, the first image is taken using a camera located overhead and the second image is taken from a camera on a rail.

In an embodiment, the first image is taken using a camera located overhead and the second image is taken either from the camera located overhead or a camera located at the side.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic diagram showing an eight part process for separating, counting, identifying and pooling insects from batches taken from traps and mapping the results according to embodiments of the present invention;

FIG. 2 is a simplified diagram showing an apparatus for carrying out the process of FIG. 1;

FIG. 3 is a simplified schematic diagram of the control of the apparatus of FIG. 2;

FIG. 4 is a simplified flow chart for the process of FIG. 1;

FIGS. 5A to 5C illustrate batches of insects from traps;

FIGS. 6A and 6B illustrate the batches of FIG. 5A to 5C being poured into a separator machine according to the present invention;

FIGS. 6C to 6E are views of the interior of a separator device according to embodiments of the present invention;

FIGS. 6F and 6G show insects being ejected individually from the separator device according to embodiments of the present invention;

FIGS. 7A and 7B are views from one side of a double cone shaped separator machine according to embodiments of the present invention;

FIGS. 8A and 8B are simplified views of the separator machine according to embodiments of the present invention looking into the space of the container; and

FIG. 9 is a simplified diagram illustrating the picking stage of picking individual insects after separation according to embodiments of the present invention;

FIGS. 10A and 10B are two simplified diagrams showing a pick and place tool according to embodiments of the present invention;

FIGS. 11A and 11B are two simplified diagrams showing an insect being placed by the pick and place tool of FIG. 10A;

FIGS. 12A and 12B are two simplified diagrams showing the pick and place tool of FIG. 10A connected to an air pressure source;

FIG. 13 is a view from above of a group of separated mosquitoes provided according to embodiments of the present invention;

FIGS. 14A to 14C illustrate placing a mosquito and taking images at different focal depths for identification according to embodiments of the present invention;

FIGS. 15A to 15E illustrate taking a series of images at different angles to obtain features for identifying species according to embodiments of the present invention;

FIG. 16 is a simplified flow chart showing a procedure for obtaining and imaging an insect for automatic identification according to embodiments of the present invention;

FIGS. 17A and 17B are simplified images showing pooling of insects in vials or test tubes according to identification using the present embodiments;

FIGS. 18A to 18H are different views including cross-sections of a pick and place tool according to a second embodiment of the present invention;

FIGS. 19A and 19B are views of screens for manual identification according to embodiments of the present invention;

FIG. 20 is a simplified view showing how the insect distribution found by the present embodiments may be displayed in map form;

FIGS. 21A to 21D are views of a separation machine according to a second embodiment of the present invention wherein the exit holes are in the floor of a separation chamber;

FIG. 22 is a simplified diagram showing an embodiment of the present invention in which insects are separated and counted on a moving conveyor;

FIGS. 23A to 23H are simplified diagrams showing further embodiments for separating, imaging and pooling insects from batches according to the present invention;

FIG. 24 is a simplified diagram showing the main anatomical features of a mosquito for classification purposes;

FIGS. 25A and 25B are simplified diagrams showing examples of insects being classified using the present embodiments; and

FIG. 26 is a simplified diagram showing a separation machine according to a further embodiment of the present invention.

### DESCRIPTION OF SPECIFIC EMBODIMENTS OF THE INVENTION

The present invention, in some embodiments thereof, relates to insect surveillance and, more particularly but not exclusively to a system and method for surveilling insect populations, including apparatus for the same.

A method is provided of separating a batch of insects from a trap into individual insects comprises pouring the batch into a container having at least one hole, the hole being sized for a single insect; and moving the container to shake the insects within so that individual insects are caused to exit via the hole onto a collecting surface, thereby providing separated insects onto the collecting surface. The insects from the trap may then be counted and image recognition may be used to identify the genus, species or gender. The process may be carried out for multiple traps and location data may be stored with the insect identifications to give a map of insect distribution.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

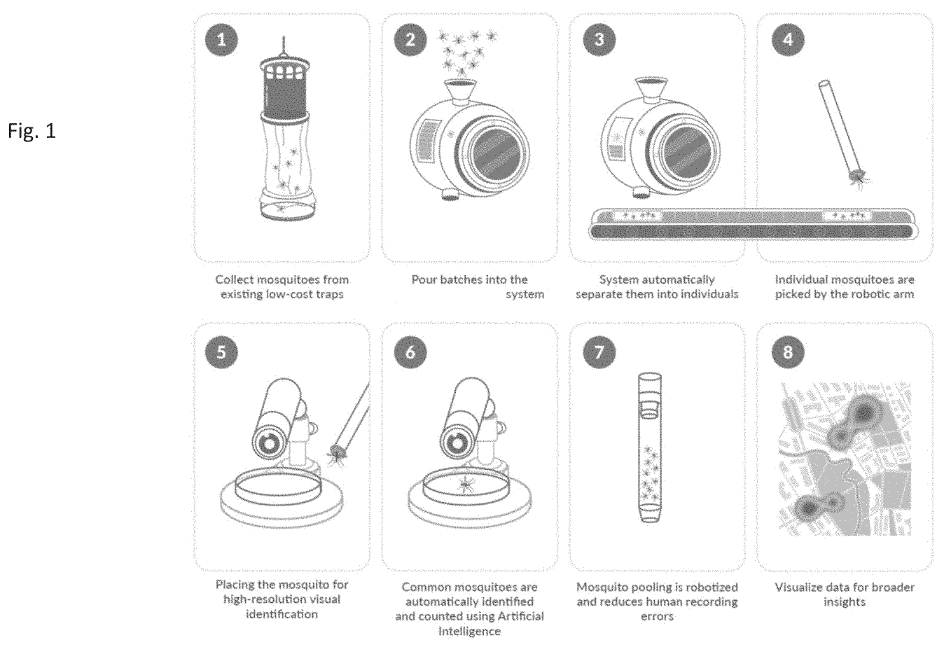

Referring now to the drawings, FIG. 1 illustrates an eight part process for monitoring an insect population according to an embodiment of the present invention, wherein the insects are caught using a legacy insect trap.

The embodiment may comprise up to eight different process parts 1..8 as shown in the FIG. 1 is used as the key for the discussion of the following figures.

The eight parts of FIG. 1 comprise a method for the automatic or semi-automatic mosquito identification-counting-pooling-mapping. Not all of the parts are mandatory. For example, having only parts 1-7, will exclude mapping only from the process, which may be sufficient in some circumstances.

In addition, there are schematic drawings of the apparatus and method, and there follows a description of the flow process, indicating what various elements in the system do.

Additional embodiments for all or part of the process are presented, it being noted that the embodiments may be mixed and part processes of different embodiments may be matched together in any way found suitable by the skilled person, who is expected to carry out a cherry-picking operation of the features found in the present description in order to match his precise requirements. As is known, field conditions may vary considerably, so that a combination that works well in one environment may hardly work or not work at all in another environment. The eight parts of the process comprise 1 collecting insects from traps. The traps may be legacy low cost traps. The insects collected may then be poured into a device according to the present embodiments, 2, which then automatically separates out individual insects, 3. The individual mosquitoes are picked out, 4, for example by a robot arm, for high resolution visual identification 6. Mosquito pooling is robotized to reduce human error, 7, as will be discussed in greater detail below, and the data is visualized, 8.

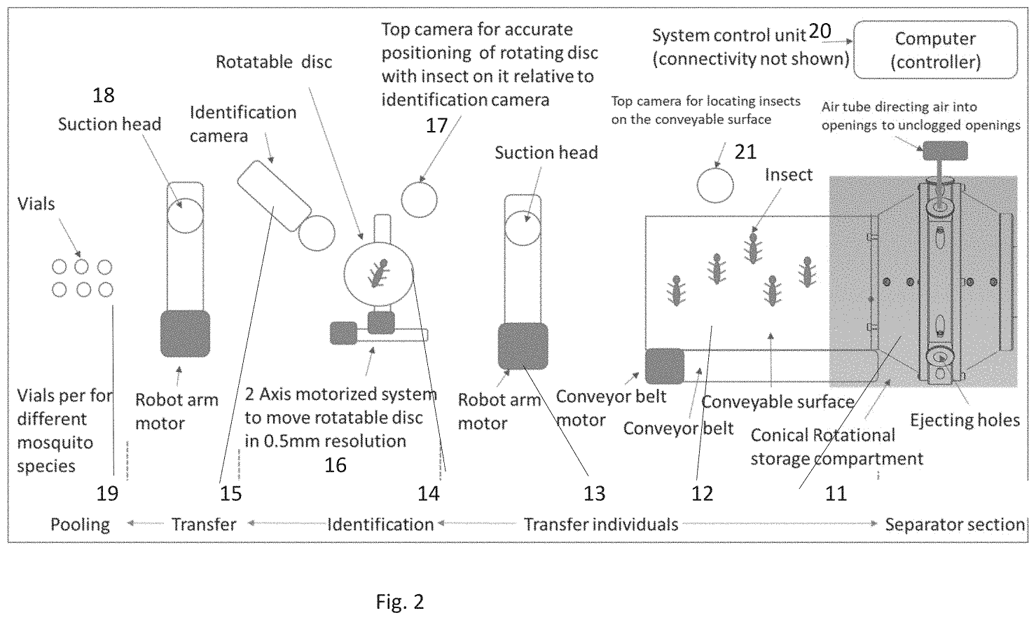

Referring now to FIG. 2, a schematic view is provided of a system that implements the method of FIG. 1. The flow process is shown in the figure from right to left. The part relating to the collection of the field trap is not depicted, and it is assumed insects were already placed inside the separator unit 11, separating mosquitoes into individuals, ejecting insects downwards as single mosquitoes onto a moving plate 12, from which a suction pipette 13 transfers them onto a rotatable disc 14, which provides constant distance and a rotational angle between the insect and the imaging camera 15. Then the position of the insect may be corrected as necessary ensuring the insect is located at a certain position with a preferred tolerance of 0.5 mm on the imaging disc, the correction being achieved by moving the imaging disc using an X-Y motorized system 16 according to the insect coordinates received from a top view camera 17. Once positioned to within a 0.5 mm tolerance, then the imaging camera 15, which may be located to the side, at say a 30 degree angle to the surface on which the insect is located, takes multiple images of the insect as the disc is rotating. When the required number of images are taken or a required number of rotations of the disc 14 are completed so as to provide views from an adequate number of different angles, then the insects are transferred using the suction unit 18 towards a bank of vials 19. Each insect is then placed in a vial matching the species of the single mosquito or other insect that has now been identified. Additionally, a general purpose vial may be designated for all mosquitoes for whom the model confidence as per their species was below a threshold confidence level e.g. 70% meaning they have not been clearly identified. The placing in vials represents the pooling part 7 in FIG. 1.

As will be explained in greater detail below with respect to later figures, the separator machine may hold a batch of insects, such as mosquitoes, inside a two cone shaped structure. The two cones are attached together into a single structure to encourage the insects to move downwards towards the middle of the two cones down the slope on both ends.

The cones may rotate together, causing insects to continuously fall downwards towards ejecting holes. Optionally, a side movement of the two cones or abrupt changes in the rotation direction may be provided to generate vibrations. As a result of the vibrations, insects may be separated from each other to fall individually through ejection holes.

The two cones may further serve as a storage compartment. If the insects are alive, the compartment can also be kept under cold conditions to support the insects being stationary or immobile and not to cling to each other in ways that may cause harm.

The separator compartment may continue to rotate to divide any large input batch of insects into small groups and encouraging individual insects to fall down and exit the ejection holes under the influence of vibration. The internal shape preferably supports the mosquitoes to keep on moving towards the exit holes, hence the preferred shape of the internal cones, however other separator compartment shapes such as those with rounded perimeters or external geometry may also be implemented, such that the continuous separation and ejection of the insects is provided.

The storage compartment surface may have one or more exit holes around the perimeter of the two cones, along their interface. It is shown in greater detail hereinbelow how the same functionality, of vibrating may separate insects and then allowing them to be ejected or fall as individuals, may be implemented in other ways, for example using a planar vibrating surface having one or more exit holes. Yet further shapes, other than the two cones or the planar surface, may be implemented by the expert, based on the present embodiments relating to separation into individual insects using vibrations and having exit holes. Such vibrations may cause separation by means of vibration and ejection of the separated individuals throughout the exit holes or openings.

Once the insects have fallen out they reach a movable or conveyable surface 12.

An issue that arises is clogging of the exit holes. As insects exit through the ejecting holes, they may cause clogging if they get stuck inside the openings and do not exit. Hereinbelow there is a discussion about the diameter of the holes and how a phase like exit shape may be used to prevent clogging. In one embodiment, there is an option to place an air source, for example air flow coming from an air pipe at a fixed location directly in front of where the ejecting holes pass, so as to puff air towards the inside of the compartment, pushing the clogging insect back in, thereby unclogging the opening. Unclogging may also be solved by increasing the vibration forces momentarily, or even increasing the size of the exit hole, by taking it out, preferably momentarily.

A top view camera 21 may identify the locations of insects on the conveying surface 12.

Once the coordinates of an individual insect on the surface 12 have been identified, a pick and place robotic arm 13 is directed to reach above the insect. The robot arm preferably moves on the Z axis towards the insect and collects the insect by means of holding it using suction. The insect is held against the suction through the tube by means of a mesh, that is a net or porous surface, towards the end of the suction tube. A more detailed description of the tube and mesh structure is provided below.

The pick and place robot arm 13 may transfer the insect towards the rotatable disc 14 which implements a relative movement between the identification camera 15 and the insect, so as to obtain a set of successive images of the same insect from different angles.

It is appreciated that the transfer stage of the individual separated mosquito may be a single stage wherein the insect is directly moved after being ejected from the separator exit hole towards an imaging location, or it can be the multi-stage process described above which includes linear movement of a surface on which insects are located and then a second transfer utilizing a pick and place robotic arm being guided by a camera directing the pick and place to where the insect is located, and moving it to the imaging location.

In order to place the insect on the disc 14, the control 20 may stop the suction of the pick tool, and the insect drops from the surface on which it is being held, the net, or porous surface with a pressure difference, as there is no longer air pressure holding the insect. In order to ensure accurate placement on the disc relative to the imaging camera, in a secondary stage, the motorized two axis motor 16 to which the rotatable disc is attached, may move the disc to a position to place the insect at an accurate location or distance relative to the imaging camera, preferably to a tolerance of 0.5 mm.

After positioning, the control system 20 may commands the imaging camera 15 to take successive images of the insect as the disc 14 rotates. The disc 15 may stop rotating before each image is taken to ensure all images are in focus and to reduce or avoid blur.

Other possibilities for transferring the insects may include letting the insect at the end of the conveyor fall down a funnel towards the rotating disc, which disc would thus be located lower down.

After generating the images, a computer vision model, preferably a neural network model, may identify unique species identifying features, as will be discussed in greater detail below, and is then able to identify the insect species based on that information.

After the insect is identified, it is transferred using either the same or another robotic arm 18, preferably based on suction or air flow, towards the bank of vials 19 and into the vial matching that specific insect type.

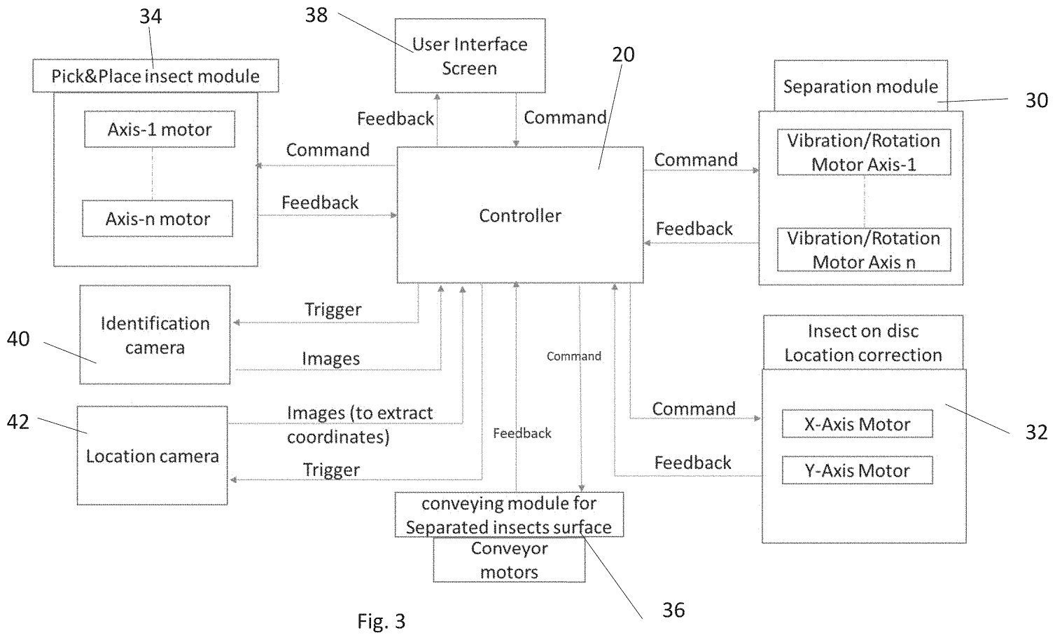

Reference is now made to FIG. 3, which is a schematic block diagram of the interface between the control system and the different modules.

As shown in FIG. 3, controller 20 provides command and feedback control to the various parts of the system and receives feedback. The parts include separation module 30 which has 1 . . . n vibration rotation axes, the rotation disc x and y axis motors 32. Additionally the pick and place insect module 34 has two single motor axes, and there may be more than one such pick and place module. Conveying module 36 operates the conveying surface.

In addition to command and control there are units that provide triggers for operation such as images or user commands, including user interface screen 38, and location and identification camera modules 40 and 42, of which cameras there may be more than one.

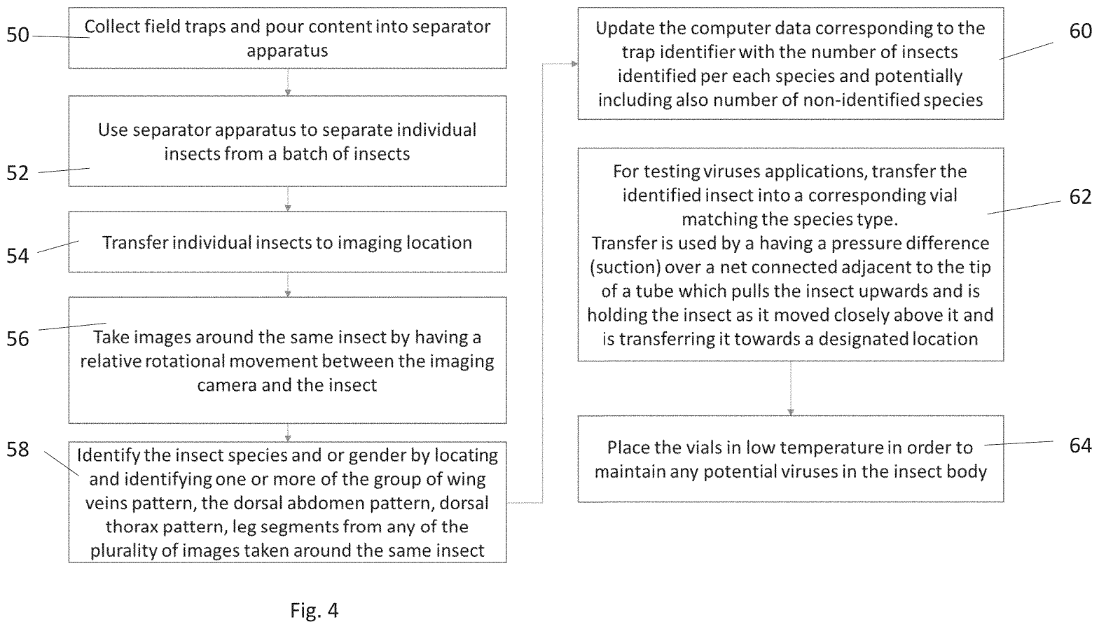

Reference is now made to FIG. 4, which is a simplified flow diagram illustrating the above embodiment.

The insects are collected from field traps and poured into the separator apparatus, 50. Individual insects are then separated out from the batch, 52. The separated insects are transferred to an imaging location, 54. Images are taken at different angles around the insect, 56. Using the images and image recognition technology, the insect species, gender etc is identified based on identifying features that are found to be distinctive, such as wing vein patterns, dorsal abdomen pattern, dorsal thorax pattern, mouth parts, shapes of mouth parts, and leg segments, 58.

The system is updated with the numbers of each identified species, gender etc. and as necessary with numbers of non-identified or tentatively identified insects, and in many cases this may be all that is required.

However in some cases, the insects, or some of the insects, may be required for further testing, for example to find out how many of the insects are carrying disease, whether viruses, bacteria or animal parasite. As per 62 the insects are collected into a vial corresponding to the species or gender identified. The vials may be stored at low temperature, for example to ensure retention of viruses inside the insect's body so that they are still present for later testing.

The process is now described in greater detail.

Insects are collected from field traps. Insects may include different types of mosquito species as well as potentially other insects which were caught inside the trap. The trap may include different insect sizes.
The trap may be left in the field for a few days, or it can be placed on the evening and be taken the next morning to ensure majority of the insects are alive. In such way, many insects remain alive which at times is important for testing the presence of vectors carried by the trapped insects (for example the mosquitoes).

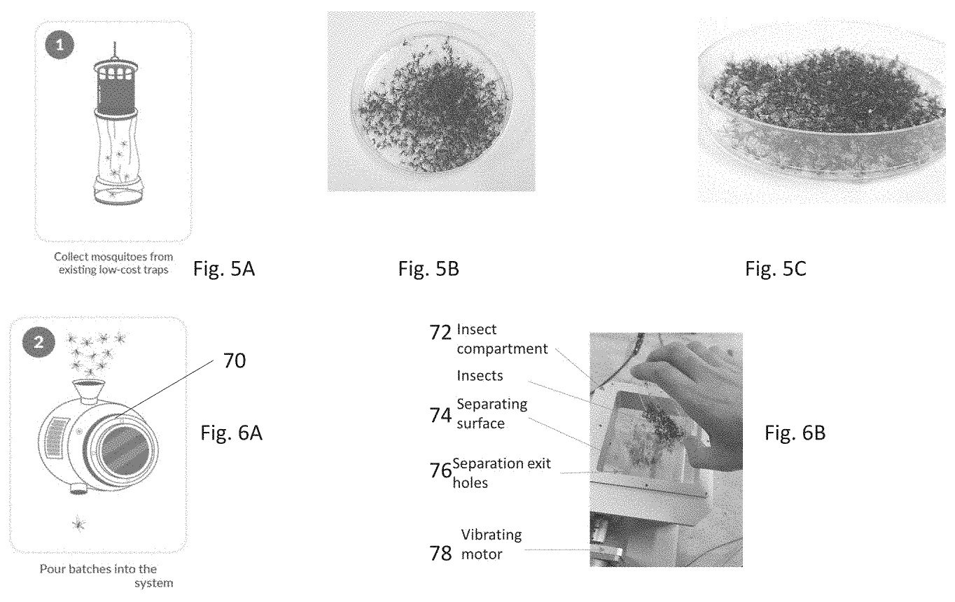

As per FIG. 5A, insects are collected from the traps, and may conveniently be poured onto a petri dish as shown in FIGS. 5B and 5C which are two different views of a petri dish containing mosquitoes of aedes albopictus.

In the current art, counting and identifying species for such a number of mosquitoes is a time consuming and repetitive task.

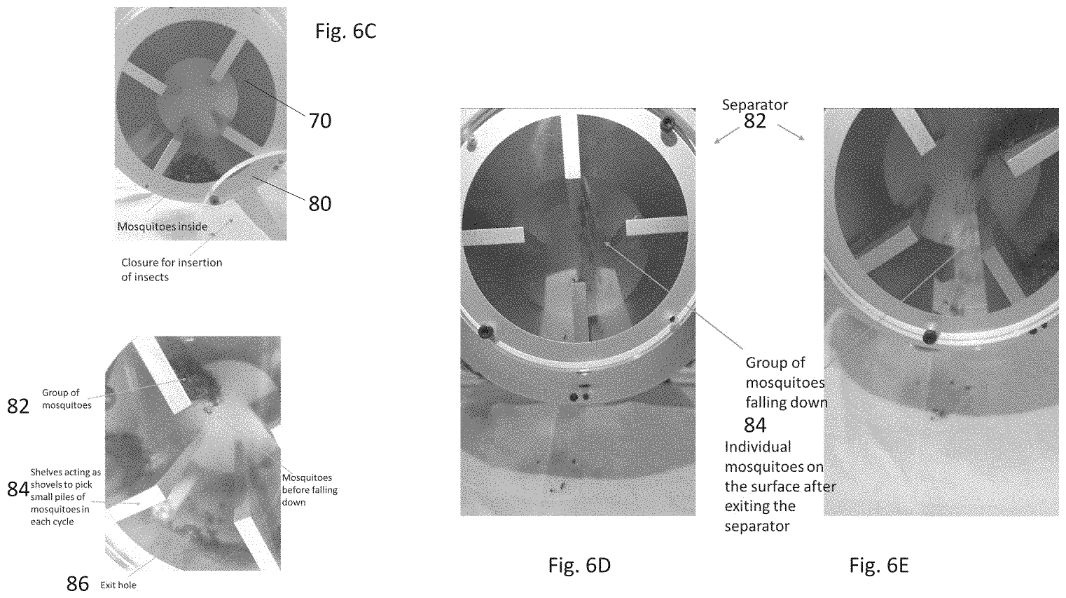

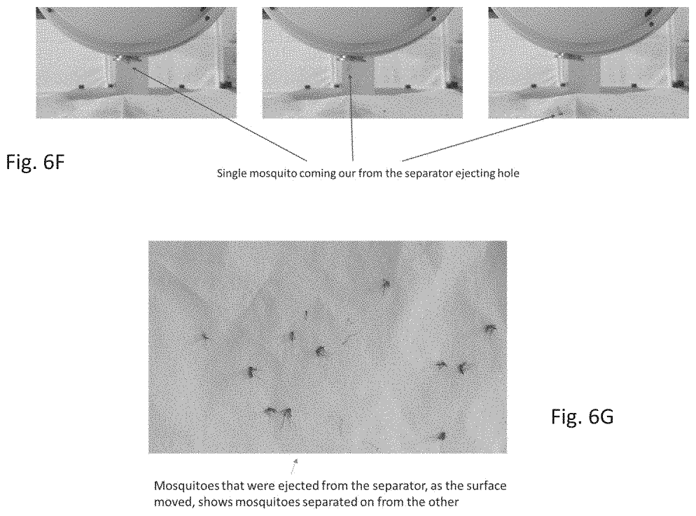

As shown in FIG. 6A, the insects are poured into a separator according to the present embodiments. Specifically, the trap content is spilled either directly from the trap, or from the petri dish into a separator module 70.

As shown in FIG. 6B, separator 70 comprises an insect storage compartment 72 which is open and ready for insertion of the insects, after which it may be closed. A group of mosquitoes is already awaiting inside for separation while more are being poured in. The module includes separating surface 74, exit holes 76 and vibration motor 78.

FIG. 6C shows the same view from above, and showing closure 80.

The module ensures separation of the batch of mosquitoes into individual insects, or at least into smaller groupings so as to allow the number of mosquitoes to be counted, identified and or manipulated. For example, at the high end, the module manages to separate each and every single mosquito, and all mosquitoes falling from the separator on the receiving surface are completely apart from the other, and are then identified one by one as shall be later descried. On the other hand, in a different scenario, for example only 25% of the mosquitoes are separated with success, meaning at times there are still insects touching each other, thus making it more difficult for a vision system to identify the single mosquito due to possible obscuring of important visual features. The user may in such a case extrapolate the results by 4, when say 25% were successfully identified, to estimate the number of mosquitoes and their species for the entire batch. While this will not be an accurate result, it does provide a result with a certain statistical reliability.

The separation module 70 as shown in FIG. 6D shows a group of mosquitoes 82 moving towards the end of a shelf-like component 84 which serves as a shovel. The shovel 84, at each rotation, picks a number of mosquitoes from the bottom and spills them downwards towards an opening 86 on one end, from which a single mosquito is ejected onto the other end of the surface.

The storage compartment 72 may rotate on its axis, so that single individuals are ejected or fall down from the exit holes.

The expert may use the present concept for the separation of the mosquitoes for other applications which require separation of mosquitoes, for example for release systems, in which a batch of mosquitoes awaiting release is stored inside the compartment and then as the separation starts, a continuous flow of individual insects is provided from the storage compartment. Such a module may be attached underneath a UAV (unmanned flying aircraft) to release insects above an area for biological control. Likewise the module may be attached to other release systems.

FIGS. 6D and 6E show successive stages in use of the separator unit 70.

In FIGS. 6D and 6E there is shown a group of mosquitoes 84. As the machine rotates, and preferably vibrates back and forth, for example perpendicular to the direction of rotation, single mosquitoes are ejected from the exit holes and received on a surface. FIG. 6F shows insects being expelled one by one, and FIG. 6G shows the individual insects being collected on a surface.

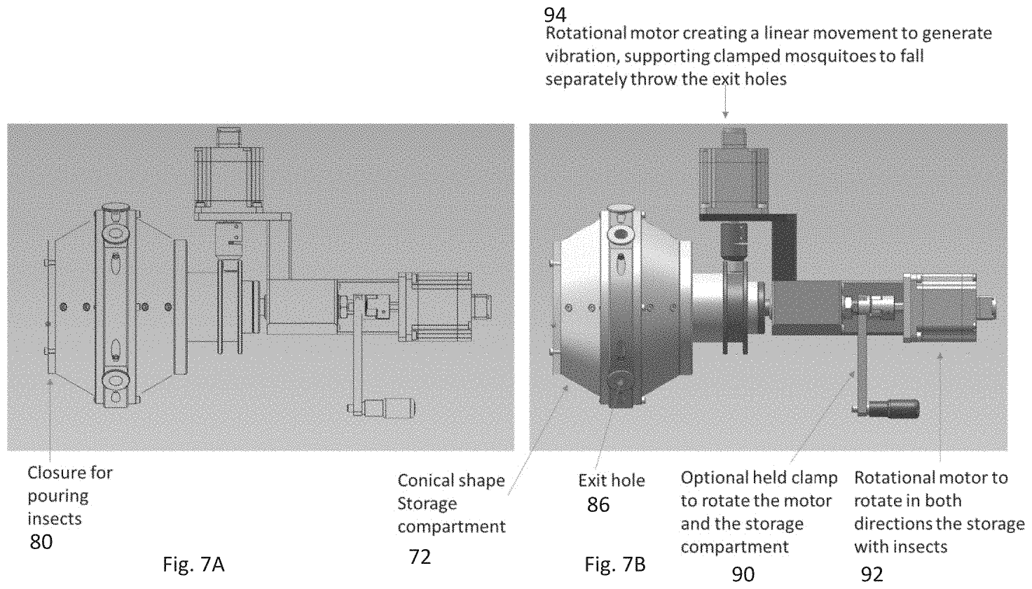

Reference is now made to FIGS. 7A and 7B, which shows flat and 3D views respectively of the separator apparatus 70 according to an embodiment of the present invention.

The exit holes 86 enable the insect to fall downwards as the surface on which they are located is vibrated. As it rotates, the internal structure of shelves 84 enables continuous separation of the larger group into multiple smaller groups so that they are fed one at a time within the compartment towards the exit hole. Closure 80 is closed after pouring the insects. The storage compartment 72 has a double conical shape. A handle 90 may be used to rotate the motor and storage compartment if needed, say when the field location lacks power, and rotational motor 92 provides rotational motion in most circumstances. A second rotational motor 94 may be used to provide a linear movement to provide vibration, allowing clamped mosquitoes to fall out.

It is noted that a rotational unit with exit holes can be implemented without the internal shelves, however it is preferred to have them, and thus the main embodiment include them.

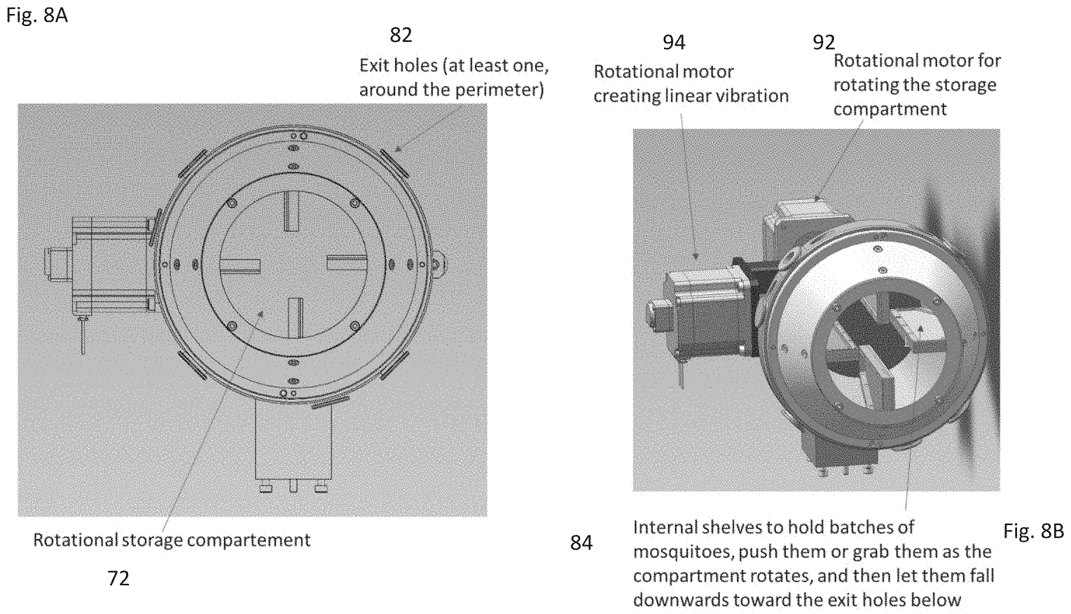

Reference is now made to FIGS. 8A and 8B, which are a cross section and a perspective view respectively of the opened storage compartment from above, and showing the motors behind.

The rotational storage compartment 72 has at least one exit hole 82 around the perimeter, and internal shelves 84 hold batches of mosquitoes, which they push or pick up and let them fall towards the holes. The rotational motors 92 and 94 for rotation and vibration are shown to the rear.
Thus, the original batch of insects is separated into smaller groups, which are picked up in small numbers by mechanical elements as the compartment 72 rotates, and as the rotation continues, eventually they fall down towards the middle section of the two cones where there is an exit hole 82, for individual ejection of the insects. Vibration of the surface if provided, by means of moving the entire drum-like shape structure back and forth at regular intervals may improve the separation process.

If the insects need to be kept at low temperature, another advantage of the above design is that as it is a relatively closed compartment, it may be kept at lower temperature, for example by introducing insulating walls to the structure.

Vibration may be provided in a cycle, for example the control program commands the motors to rotate the storage device clockwise and after a cycle to momentarily add a vibration in the axis perpendicular to the rotation, causing the mosquitoes near the holes to pass through the hole, thus being be separated from other mosquitoes to which they may have been attached.

Speed of rotation, duration of vibration and its direction, and other parameters may all be altered to optimize the individual separation for the mix of insects obtained.

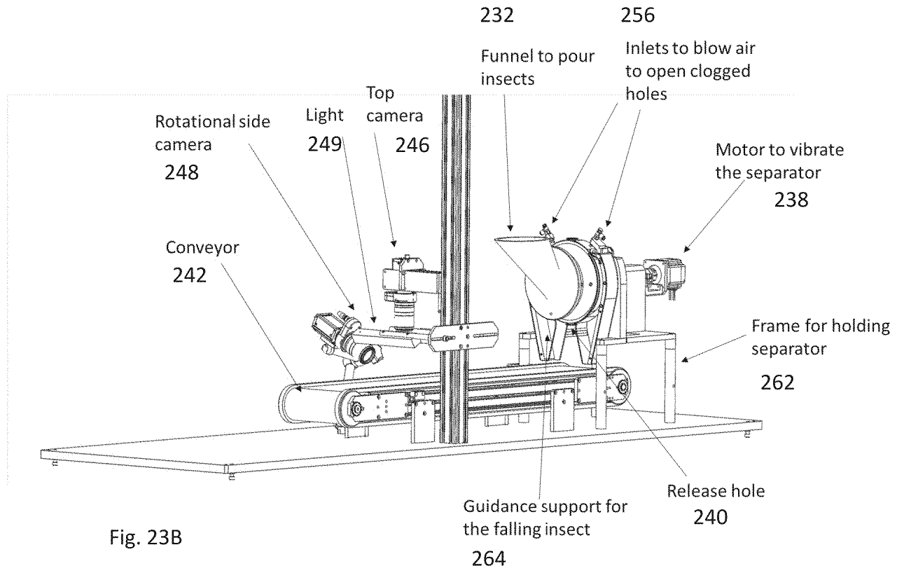

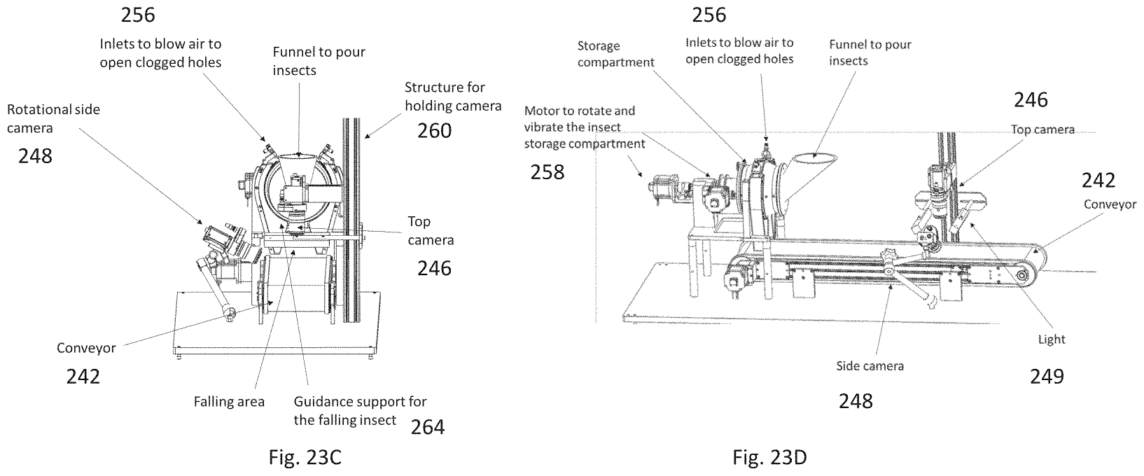

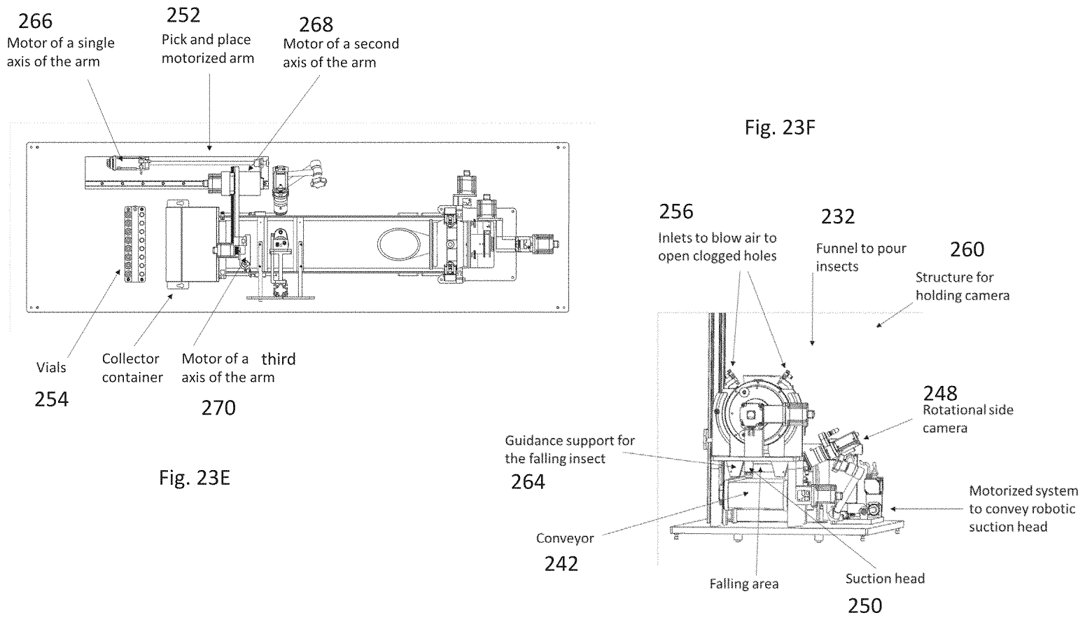

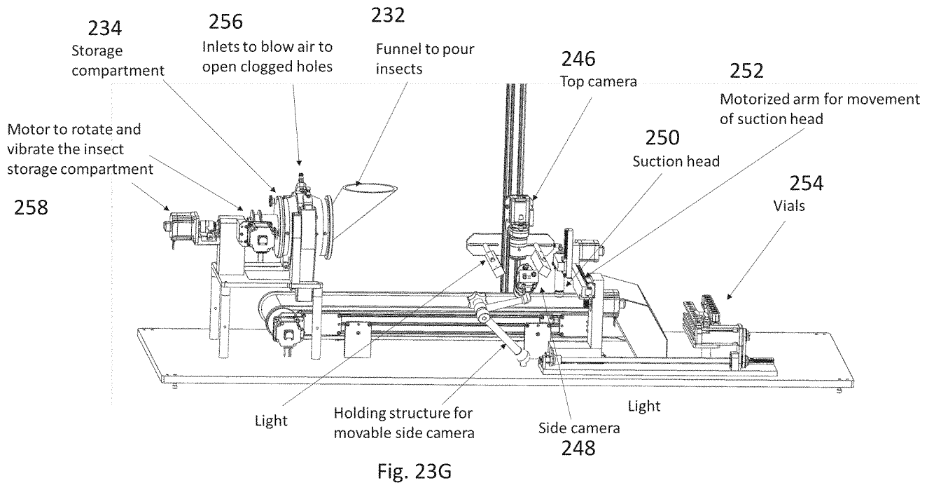

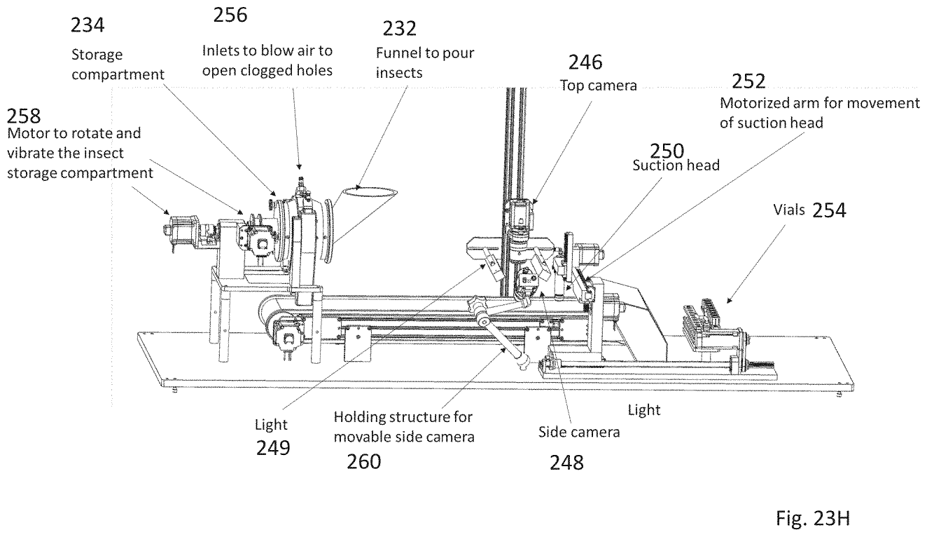

As the drum rotates, an air flow pipe may clear any potential clogging of the exits from the separator unit, as shown in FIGS. 23B-H below.

An alternative embodiment for avoiding clogging comprises the creation holes that are narrow on the inside but widen outwardly, ensuring that no insect too large to get through will manage to get in. For example, the entrance side may be 4mm and the exit hole 6mm, or as suitable for the insects in question.

It is noted, that the rotating compartment has heretofore been described as drum shaped and may have zero or multiple shelves to grab insects as the compartment rotates and let them fall back toward the floor of the drum and then to exit from the exit holes. The drum may have exit holes of different sizes, allowing different size insects to be deposited. The drum may be rotated clockwise and counterclockwise during the operation (for example 1 cycle clockwise, and then 1 cycle counter clockwise, or any other combination).

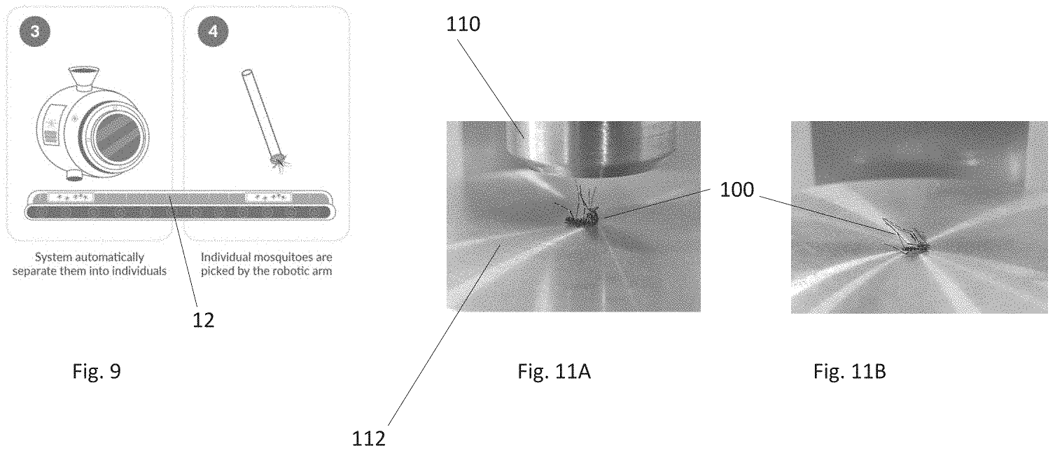

Once mosquitoes are ejected from the separation module, they are transferred towards an imaging station in a transfer stage shown in summary in FIG. 9. The insects land on a surface on which they are conveyed and/or are picked up by a pick-up unit on a robot arm and transferred to the imaging location.

Specifically, the ejected mosquito 100 falls onto conveying element or conveyable surface 12. It can be a conveyor belt, or it can be a pallet placed on a conveyor belt (or other conveying mechanism). The surface on which it lands is preferably synchronized with the operation of the separation module 70.

Transfer may be carried out in batches. Thus each time a batch of a preset number of mosquitoes is present on the surface, a transfer is carried out. Alternatively, a single mosquito is transferred each time.

A pallet may be located above a conveyor to receive small numbers of mosquitoes directly as they are ejected from the separator unit.

In order to avoid piling up of insects on the pallet, the pallet may move away from the separator as it is being filled. The speed of movement is adjusted depending on the rate of the falling insects. A line of insects may be formed using a conveying element moving at a suitable rate.

In the present embodiments, the rate of insect ejection is related to the rotational velocity of the separation module which ejects mosquitoes as it rotates.

The conveying surface may for example move the insects just collected say 30 centimeters sideways from the center of the position where they fall, after which they are taken away for the imaging process.

A location camera 21 is located above the surface on which a mosquito or mosquitoes are located, such that it can provide information pertaining to the coordinates of the insect/s on the surface. The coordinates are then sent to a pick and place unit 13 which picks the single mosquito and places it at the imaging station, hereinafter also the identification station. The identification station is where the insect is being identified for its species and or sex, either automatically or manually.

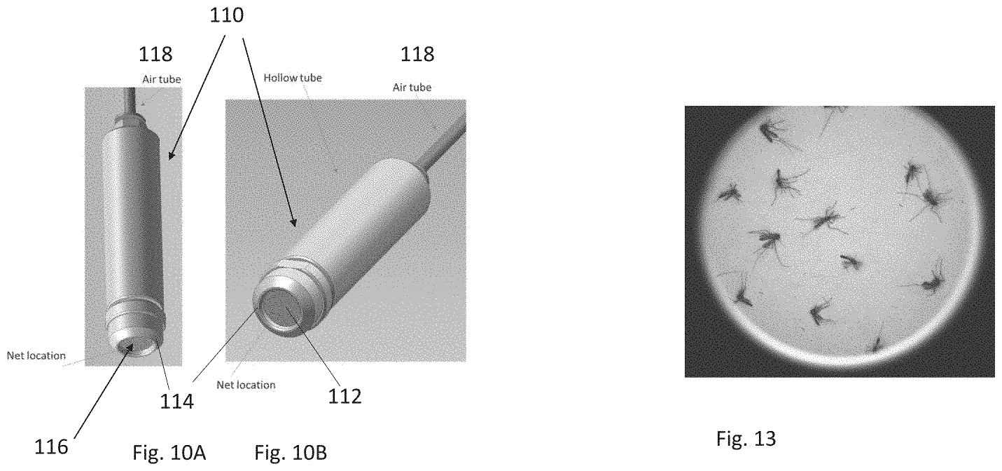

Referring now to FIGS. 10A and 10B, a pick and place element comprises a tube 110, having a porous surface 112 such as a net, located just behind the end 114 of the tube, to form a receptacle 116. An air tube 118 connects the tube to a vacuum source. In use the tube approaches the insect and sucks it up so that it is held against the net.

FIGS. 11B and 11C show the tube 110 connected to vacuum generator 120 via air tube 118. At end 114 is located the net 112 which enables a relative free passage of air flow, but does not allow the insects to get through. The size of the holes in the net may be selected for the kind of insect being surveilled.

The pick and place element 110 is connected to a moving element on at least two axis (X and Y) to be postioned just above the mosquito. Then a piston or other movement on the Z axis, lowers the tube to suck up the insect onto the net.

The pick and place element, may now be moved to transfer the one or more insects picked up, from one point to a second point.

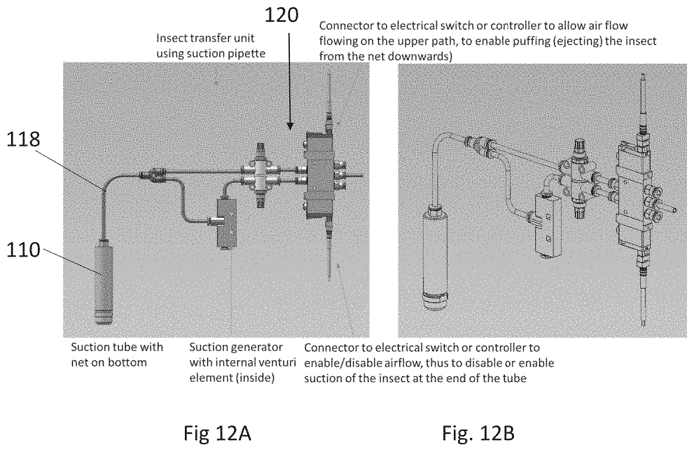

FIGS. 12A and 12B show how the pick and place element 110 drops a single mosquito 100 onto an imaging surface 112. FIG. 12A shows the element immediately after dropping the mosquito, and FIG. 12B is just afterwards, when the element is pulled up and the insect is left for identification.

As noted above, the net is not at the end of the tube of the pick and place element. Rather, the end of the tube may be some distance, say 2 mm, below the net, in order to damaging the insect while approaching the surface on which it is to be dropped. The net may alternatively be located at the very end of the tube, in which case the insect is released prior to reaching the surface, or the net may be higher up in the tube, enabling the pick and place element to actually reach the surface and thereby ensuring accurate placing on the imaging disc.

Thus the pick and place element comprises an air pipe having a net on one end and connected to a valve on the second end, to pick up individual insects.

A switch may controlled by the controller software to alternate the air pipe between suction mode or puffing mode or off.

In suction mode the element sucks the insects and causes them to be firmly held just below the net cover. While in puffing mode, the insects fall off from the net, as now air flow is directed within the pipe towards the net and outside. It is noted that having the puffing mode is not mandatory, and upon switching to off mosquitoes that were held to the net may now fall downwards.

In order to pick the insect using suction, its coordinates are identified (for example by location camera 21 and provided to a moving arm holding the suction unit.

As noted earlier an alternative to pick and place using suction, may comprise the transfer of insects from the surface on which they fell from the separator and to the identification location, by letting the insects fall down a funnel at the end of the conveyor, hence falling directly onto the imaging location.

Once the insect is placed, then in an embodiment, an enhanced accuracy positioning system may use a two-axis movement to ensure the insect is located each time at the same position relative to an imaging camera.

In an embodiment, to drop the insect as close as possible to the surface of the identification area, then, depending on the size of the mosquito as seen from above by camera 21, the height of the individual insect may be estimated to determine how high above the surface the insect needs to be released to avoid damage. The distance is preferably minimized so the insect is placed as accurately as possible on the surface.

Reference is now made to FIG. 13, which is an image of separated insects of various sizes. For the largest insect in the image, the tube may approach at a height which is higher than when it is approaching the smallest insect in the image in order to pick up the insect. The same is done when placing the insect, and an advantage is that when dropping the insect on the surface, if air flow is used to puff the insect downwards, then a minimized distance reduces the chances that the insect will be blown sideways and thus inaccurately positioned. Additional correction means may be provided to move the disc on which the insect is located, so the insect is positioned at a fixed location relative to the identification camera.

Immobilization, in various forms, may be applied in order to keep living insects in position. For example, cold air or CO2 may be applied, say to the area of the storage compartment, or the entire separator may be in an enclosure at low temperature. Alternatively, the insects may be immobilized on particular surfaces, for example using pressure difference (suction) under the conveyor 12, thus making sure live insects do not walk away.

The placement of the individual mosquito onto the imaging location is carried out in order to facilitate accurate imaging and successful identification.

As the pick and place element 110 moves to the placing position, a valve, operated by the system controller, is switched so now instead of suction, the direction of air flow is downwards, down and away from the tube, so that the insect that was held by suction to the net, is now freed and falls down.

As noted above, puffing away the mosquito is possible but not mandatory, and the mosquito may simply fall once the suction is turned off.

The placement of the insect onto the imaging location may be accurate to the order of 0.5 mm, in order to ensure that the entire insect body is in view and in focus. Meaning, it is possible to choose a camera sensor and a lens and position the camera and its lens at such a distance from the mosquito that the majority of the insect body is in focus, with a depth of field around 0.5 mm, hence if the object or parts of it (e.g. edges of the legs) is positioned more than 0.5 mm away from the center of the focus, it is no longer in focus. Hence it is desired to be able to position the object at such accuracy.

It is noted that in order to manage and image the different features of the insect, while it is preferred to have it all in focus, it is not mandatory in order to implement the solution described, and indeed in some cases the insect may be greater in size than the depth of field of the chosen optics. In such a case a set of images, each at a different focus, may be generated, for example taking sets of images each at a different focus. The sets of images may then be reconstructed to create a single focused image of the insect body, or even a 3D model. It is also possible to look for the different unique feature used to categorize the insects and set the focus to that which shows them best, however this may slow the process.

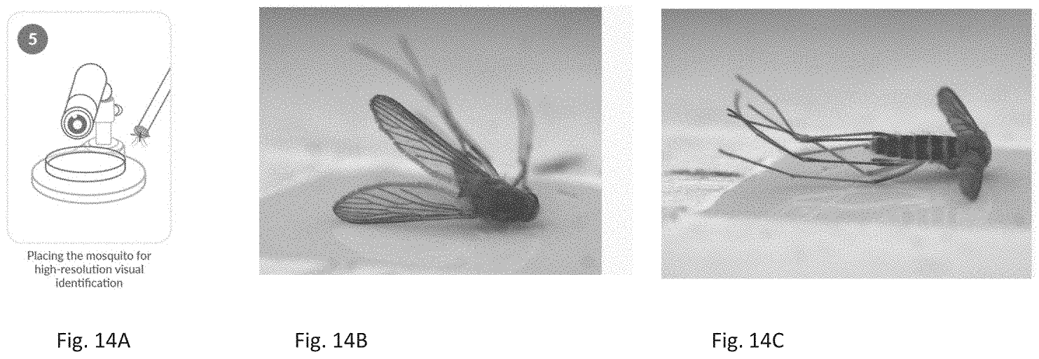

FIGS. 14B and 14C show an example in which a mosquito is rotated and then photographed with a different focus each time on a different area. Specifically, FIG. 14B focuses on the wings and FIG. 14C on the abdomen.

The level of accuracy of positioning may depend on the optics being used by the identification camera, so that 0.5 mm is merely a guide. It is now explained how the mosquito is positioned at a 0.5 mm resolution.

Initially, the suction tube holding the mosquito moves to a predetermined position and stops the suction and/or puffs the mosquito gently while the net is not so close to the surface to squash the insect but on other hand is close enough to prevent movement along the surface as the insect is detached from the holding surface.

Top view camera 17 is located to view the imaging location, and may send the coordinates of any identified object on the imaging location to the control software 20.

The control software in turn sends a correction command to the motors of the two-axis motor 16 of the disc 14 at the imaging location, to move either in either axis, so that the mosquito located on the surface is repositioned at the desired location within a 0.5 mm tolerance.

In another embodiment, first a robotic arm receives the coordinates of the insect on the surface onto which the separated insects arrive. The robotic arm may hold a camera to locate insects on the surface. Alternatively, another camera may provide such images in an intermediate stage, and the robotic arm then rotates around the mosquito generating the multiple images from different angles around, and even above, the insect. As such, by use of the robotic arm, the requirement to have a specific distance between the object and the camera sensor or lens is obtained, only using a different implementation.

Imaging may thus take place at various angles around and additionally but not necessarily, from above the individual insect. Automatic pooling now becomes possible as the mosquitoes are identified and are manipulated at the individual level so that all individuals identified as belonging to the same species, gender etc. may be placed together in the same vial.

In another embodiment, the camera, being connected to a robotic is rotated around each of the different insects located on the surface after being separated, without the need to transfer them to a designated identification station. In that case once identified the insects may be directly moved from the surface to the appropriate vial, if automatic pooling is also required.

Once the insect or insects have been imaged, the surface onto which they are received after separating is cleaned of the insects to allow a new batch of insects to be ejected from the separator unit and received on the receiving surface, which in this other embodiment is also the imaging surface. This particularly applies when collection or pooling is not required, or is not required for all the insects found but only for certain species, but may always be needed say for part specimens or bits of dirt. Cleaning is applied by having a robotic arm picking and removing individual insects with a suction pipette, or using a blower to blow on the surface and blow away any objects on the receiving surface. Hence, insects are removed from the surface using air flow.

Such cleaning methods for blowing air onto the receiving surface is applicable for all of the embodiments herein.

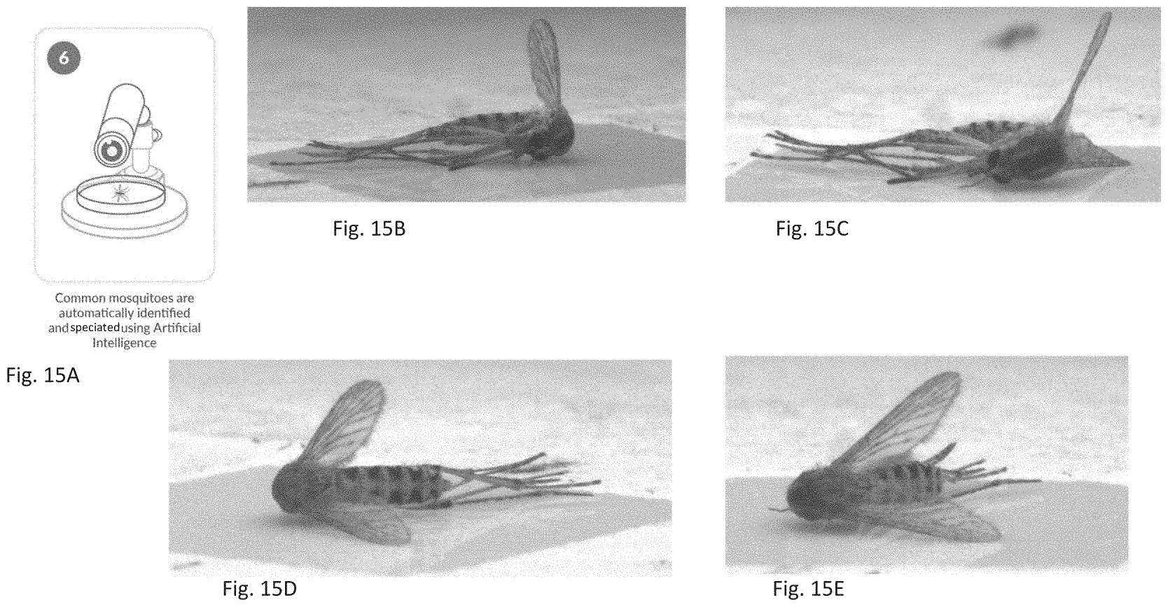

Reference is now made to FIG. 15A, which shows the images being used in a classification process to identify the species etc. Classifying may be carried out using a trained neural network using three or more layers.

The method includes obtaining images, for example from successive frames, at different angles or different perspectives, of the same insect, to determine if specific features are present at any or more of the images.

The method includes obtaining a plurality of insects, imaging the insects, and from the imaging identifying at least one from the group of the wing veins pattern, the dorsal abdomen pattern, dorsal thorax pattern, leg segments, and thus identifying the species of the insect.

As shown in FIGS. 15B to 15E, the features described above are able to be visualized due to rotating the insect after separating it and imaging it at different angles and different perspectives, ensuring viewing at least one of such feature. Different views show grayish stipes on the head area, the grayish hairs near the head area in this specific case, and different unique features on the thorax, abdomen, etc. which help identify the mosquito species

As discussed, the mosquito is imaged in multiple images, from different angles, by rotating the surface on which it is located, or more generally by creating a relative movement between the lens and the insect. Imaging may thus also be implemented by moving the camera around the insect. A further possibility is to move the camera 180 degrees around the insect and rotate the disc 180 degrees, in order to make the entire machine more compact in space. Once the images are available, a computer vision algorithm identifies the species by locating part or all of the features in the body parts from the multiple images.

As the disc is rotated, images may be taken from a fixed camera looking at a specific angle (e.g. 30 degree above the surface) at the insect. In an embodiment, the disc stops rotating before each image. The disc may for example be rotated in increments of 45 degrees, thus generating 8 different images. Different angle increments are possible. Per each angle, either a single image can be taken, or multiple images at different focus can be taken.

The images may subsequently be sent to a computer which identifies the genus, the species or the gender as desired based on a vision model. That is to say, in the case of disease control, it is the presence of specific species that tends to be of interest and often of a specific gender. For general environmental surveillance, the population mix is of interest and data may be required at any level of detail, such as genus.

The computer vision may as mentioned use a neural network, and the network may implement a trained model. Training may involve a human operator tagging one or more insects of the genus, species or gender of interest, the tag specifying which mosquito species it is.

One embodiment provides a method of insect identification for release and capture operations. Insects are released with fluorescent marking and the camera may be set up with a light that activates the marking and which can subsequently be identified. The scene for the camera may be lit using a wavelength chosen to excite the fluorescent markers on individual insects coming from the trap that had earlier been released and marked. Such data is valuable for researches trying to learn about flying distance and other behaviors of the insects they have released.

After the identification process, if samples are needed, the insect may be transferred again, preferably into a specific vial, or tube or storage compartment designated to hold that specific species.

Insects not successfully separated or not successfully identified may be dropped into a separate vial to include all such non-automatically-identified or non-separated insects.

The number of rotations and the angle of each rotation may be pre-set, for example with an image at each 15 degree. Also, one or more of the images may be taken from above the insect, that is above the rotating disc, the software guiding the camera as per the exact coordinates of the insect in a pre-defined coordinate system. The image from above may be used to provide information such as the posture of the insect, respective locations of the head and the abdomen, and based on knowing where the unique features are, the software may guide the system to move the camera or the disc, to those locations and expedite the process. Such a process may be instead of taking an image every 15 degree for example regardless of the insect posture.

As discussed above, in cases where the camera depth of field enables viewing only part of the entire object in focus, as the object width is larger than the depth of field, a set of images with different focus are obtained to ensure having sufficient data of the different features of the object, as shown in FIGS. 14B and 14C. When only part of the image is in focus, the analysis process is eased, since the parts which are not in focus can be ignored.

In the identification process, it is possible to crop the different body parts, and the different body parts that are identified, such as head, abdomen, wings etc. may be sent individually to a vision processing model such as a neural network that is trained to identify the species based on body parts alone. For example, aedes albopictus has a very distinctive white stripe on his head, so an algorithm may identify and crop the area of the head, and then analyze the head image, by locating the white stripe.

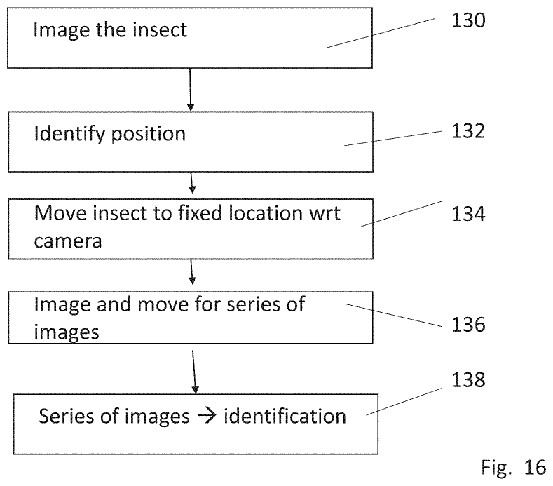

Reference is now made to FIG. 16, which shows the procedure in one possible embodiment of the identification process. An initial orientation image is taken—130—to locate the insect and if required to determine its orientation—132.

The insect or camera is moved to get the insect to a desired initial location relative to the camera—134. Then one or more images are taken and the camera moves to successive imaging positions to take more images, eventually obtaining a sequence of images—136.

After imaging the features, the images are sent—138—to a model which provides a score for each image, to which class (species, gender etc) it best matches. An overall score over the sequence of images then gives the identification result for the entire set of images, identifying the insect. If the mosquito is not identified with high enough confidence by the computer it may be labelled “other” or “unknown” for later manual identification by an operator, who may manually tag such all unknown images.

In an embodiment, the controller may guide the robotic system to move the camera or disc until one or all of a set of features are present in the images.

Considering the model in greater detail, in an embodiment, for a given mosquito top view image, the goal in a realtime machine is to reduce significantly the number of side view images required to correctly detect the mosquito species. Deep learning models may be used to exploit the mosquitoes's morphological features in a way that is similar to those used by human experts to identify the mosquitoes.

Images of mosquitoes with certain postures and body parts, such as flatbed wings legs, abdomen, thorax, proboscis, are required to achieve good classification performance.

Finally, by acquiring only few optimal side views one can improve significantly the machine performance, and decreasing both time and memory consumption.

It is a challenge to classify mosquito species having high inter-species similarity and intra-species variations.

One approach is to prepare a sparse typical top view and a few side view pictures that covers various postures/pose/view angle and with certain body parts.
For each vector mosquito species, we acquire sparse N top-view images with various postures/pose/view angle and with certain body parts.
N=a number of typical top views with different posture/pose/view angle mosquito body. [estimate N˜2-3 dorsal, 2-3 lateral, 2-3 ventral]

For each top-view posture/pose mosquito body, one can manually rotate the camera by 45 degrees and take a picture. There may be summing of up to 8 top view images.

For each original top-view, without rotations, one may acquire M side view images with significant morphological features, such as wings/ legs/ abdomen/ thorax/ head etc., required to achieve good classification performance.
M=a number of typical side view images, with significant morphological features, such as wings/legs/abdomen/thorax/head see features table file
these side views are taken only once, since it's the same for all 8 top rotations. [estimated M˜3 side view images]

Side view angles may have an elevation angle fixed at 27 degree above horizontal. Azimuth angle range is 0, 360 degree. Tilt angle is fixed at 0 degree.
Object-camera distance is fixed.
Each acquired indexed image is labelled by the following parameters:
top or side view
top with body posture and camera relative pose its azimuth angle viewed [in case of 8 views; 0, 45,90,135, 180, 225, 270, 315 degrees] where body posture is dorsal/lateral/ventral, as mentioned in features table file
side view with orientation=(elevation, azimuth, tilt) angles, camera-object distance and its typical organ [wings/legs/abdomen/thorax/head]. In our case, tilt is 0, elevation is 27, so the orientation=(27, azimuth, 0) and distance is fixed

For a given top view image, the model may find the best match with the database top-views, and as a consequence get the side pose-views with the unique features.
Next, the camera is moved to optimal positions and orientation and acquires images around the insect.

For a given insect we may take a top view picture [source image]
The model then finds a best top view match by running a similarity algorithm on all (source, destination) image pairs, where the source is a current input top view image and the destination is any top view image in the database. For example, suppose there are two species, with 64 top views each, the total number of match pairs is 128. This can be run in batch/parallel, and the final outcome is the best top view with the highest probability match.

From the best top view, one may extract the optimal side view orientation/distance from a labelled database.

For example, suppose the best top view is top lateral with angle 45 degree, but in the database we have optimal side views that correspond to a top view at lateral 0 degree.
So, one adds 45 degrees to the azimuth in the side views, in other words, one takes side views where the orientation center is (27, 45, 0) and one image is to the left and the other to the right. The outpuput is the best one top view image and its corresponding 3 optimal side views images.

Finally, for genus/species/gender classification we use the acquired top-view and three optimal side view images.

A second approach uses deep learning.

For a given top view image, one finds the best match angle view[dorsal/lateral] and posture/pose-views with the unique features.
Data is initially prepared for training.

For each vector mosquito species, acquire sparse N top-view images with various postures/pose/view angle and with certain body parts.

Each acquired indexed image is labelled by the following parameters:

Each image is labelled with the angle view dorsal or lateral or ventral.

Each image is labelled with body skeletonized polyline from tail to head/palps/proboscis. This skeleton represents the angle posture. For example, the tail is at coordinates (5,4), head coordinates (23, 20) and then the polyline is {(5,4), (23, 20))

Each image is labelelled with significant morphological features organs, such as wings/legs/abdomen/thorax/head etc.

Each image is labelled with optimal side view parameters and with genus/species/sex N= at least 10,000 labelled images

The algorithm learns to extract body posture-pose [3D shape] from top view image [2D image].

The real time process predicts a best posture-pos-view, and extracts optimal side views from labeled parameters. Side view images are acquired. Finally, for genus/species/gender classification use acquired one top-view and optimal side view images.

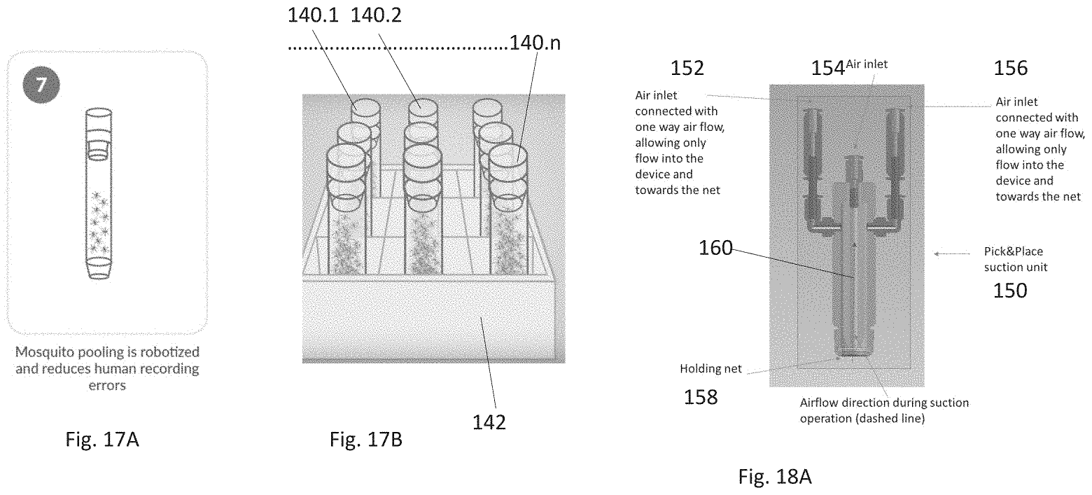

Reference is now made to FIGS. 17A and 17B, which illustrate pooling, namely placing the mosquitoes in vials for later inspection or testing, for example for virus testing. Pooling is an option, and may be dispensed with if not required, so that once a mosquito is identified it may simply be dumped, for example by rotating or tilting the surface on which it is located so that it falls off, possibly guided by air puffing from the side, or the suction tube may pick it and remove it to a common removal position which is the same for all.

However, when pooling is required, then for implementing the automated pooling process, a number of vials 140.1 . . . 140.n are located together in a location 142 such that a moving arm picking the mosquito from the imaging location can bring it to the location 142. Precision control, say using a Z axis motor, for example pneumatic using an air piston or an electrical piston, enables movement towards the vial and placing of the mosquito into the vial by puffing it inside the vial and or by shutting off the suction. In an embodiment, all axis movements can be implemented by a multi axis robot such as an articulated commercial robot.

Each vial 140.1 . . . 140.n may be assigned a unique identifier, for example using a barcode, and mosquitoes from the same species may be transferred into the respective vial, such that by the end of the pooling process, there are a set of vials with mosquitoes (e.g. 50 mosquitoes per vial) of the same species per vial, or the same species from the same location.

As mentioned, there may also be another general vial to which all mosquitoes that were not identified automatically may be transferred into for later additional analysis by other means. When pooling is required, then the machine or at least the vials may be placed inside a cooling area, ensuring a preferred temperature of 4 degree Celsius, more generally a temperature close to freezing but above it, to allow smooth operation of electronic parts, the robotic arms, motors, cameras etc.), to ensure the specimens are kept cold for say virus testing. In one embodiment, the entire solution including the separator, the transfer conveying element, the imaging station and the vials may be kept inside a closure in cold conditions preferably with an active cooling system. Alternatively, only parts of the system which keep the insects on or in them are kept cold, such as the separator unit, the conveying system, the imaging station or just the vials.

Holding of the insect and moving it from the identification station to and into the corresponding vial may be implemented by different methods that may suggest themselves to the skilled person.

One method is to use a pick and place device as described hereinabove, in which a pressure difference is applied across a net at the end of a suction tube to hold the insect while a robot arm moves the tube from the pick position to the place position.

As the tube reaches the coordinate of the corresponding vial, it may move downwards, and as it is located above the target vial, it may shut down the pressure difference causing the insect to fall from the net. Optionally, the pressure difference may be reversed, and air flow may puff the insect towards and into the vial.

The tube holding the insect may be lowered as close as 1mm above the vial opening, or until it is almost flush with the opening surface. In embodiments the tube diameter is smaller than the vial opening, in which case the tube may enter the vial and then drop the insect.

In a further embodiment, instead of or together with air flow, a mechanical gripper such as mechanical tweezers may hold the insect and as the gripper is located above the vial, open the gripper and drop the insect into the vial. Such opening and directing of the mechanical gripper is controlled and managed by the system controller.

The entire system or parts of it may remain under cold conditions to support immobilization of live organism as well as supporting storage conditions for dead organisms preserving any potential viruses in them.

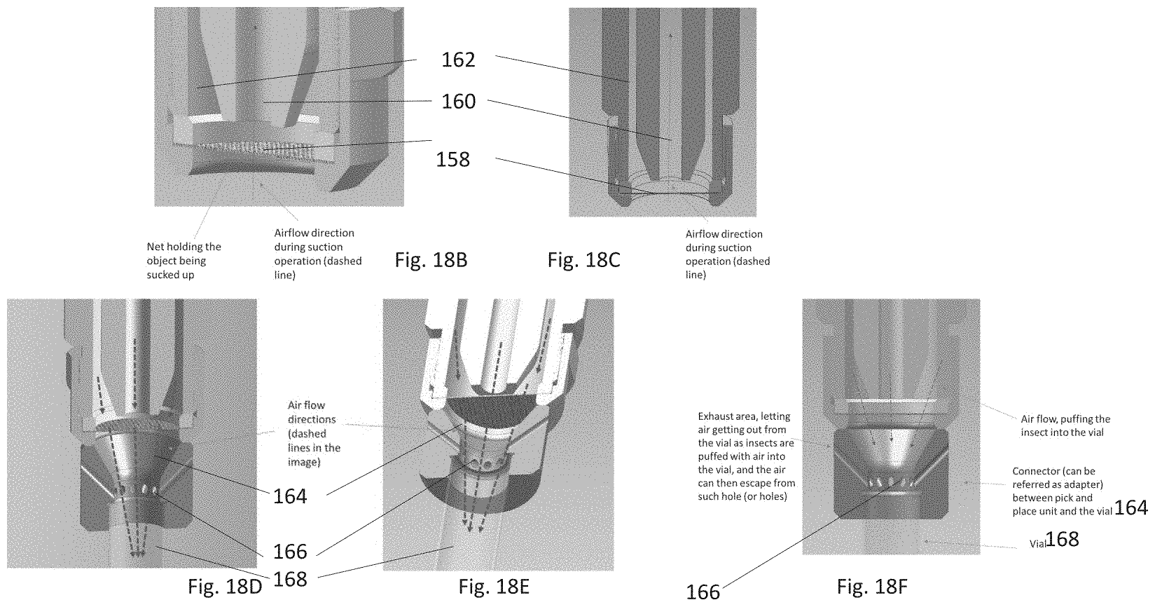

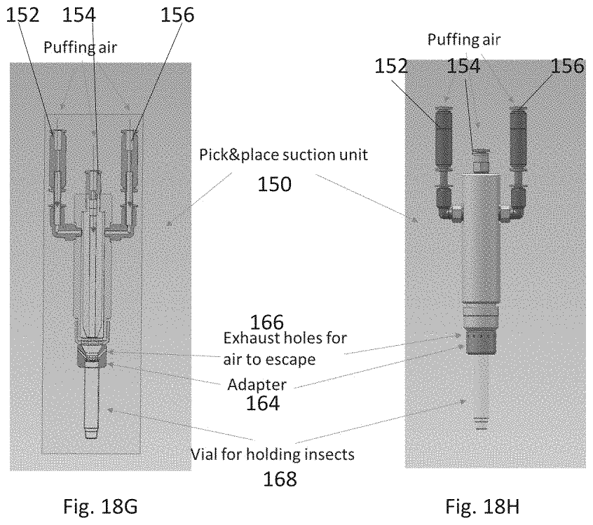

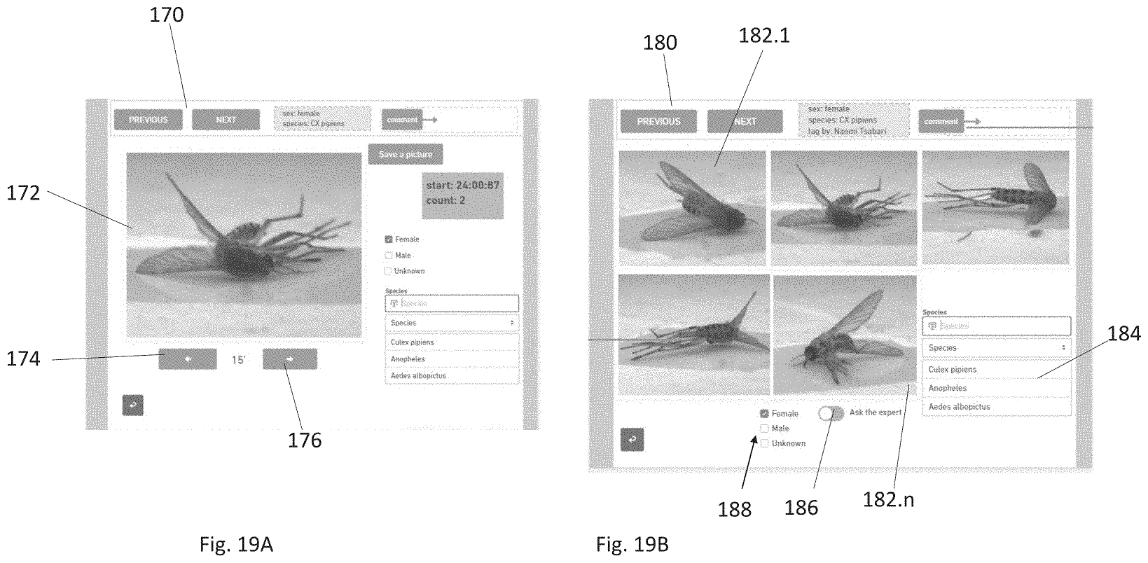

Reference is now made to FIGS. 18A-18H, which show an alternative implementation of the suction tube. As explained the insects are transferred into the vials, and the tube may use an adapter, also referred to herein as a connector, between the vial and the suction pick and place unit, referred to herein as the tube. The adapter may be part of the vial, or may be placed on top of the vial, or may be attached to the suction unit as required. For example dropping the insects into the vial from a height the adapter may be attached to the tube, as will be shown in greater detail hereinbelow.

The pick and place tool based on a suction arm picks up the insect, transferring it and then placing it at a target location. The insect may be fragile, and mosquitoes are fragile, and handling may require care not to crush the insect.

Referring now to FIG. 18A, a tube 150 attached to air pipes 152, 154 and 156 is shown in longitudinal cross section. During suction, air flow is from the middle area of the net at the bottom of the device, the holding net 158, holds the insect as possible to the center of the net. Air flow during suction is through the center 60 of the tube 150, as only central air inlet 154 allows for suction, even though all three air inlets, 152, 154 and 156 are connected through air pipes to vacuum generators. The air flow does not flow outside of the middle area 160, that is to say through outer passage 162, because the air inlets on both sides 152, 156 have one-way air flow valves, allowing air flow to flow only downwards, towards the net. FIGS. 18B and 18C show the area around the net in greater detail.

Thus, when the mosquito, or sand fly or other insect of interest is to be placed in a vial, then air flow is reversed to be directed downwardly towards the net. The three air inlets 152, 154 and 156 are used together, perhaps connected to the same outlet from the vacuum source. Air flows downwards through all 3 inlets and puffs the insect off the net and into the vial.

Connector 164 facilitates placing of the suction unit to touch the vial 168 when puffing air, and includes exhaust holes 166 to allow air to escape. The exhaust hole diameter is smaller than the smallest insect likely to be of interest for example 0.5 mm diameter, 1 mm diameter, 2 mm diameter etc. In embodiments, all holes are of the same diameter, for example, all of them are 0.5 mm).

If connector 164 is not used, then if the tube is placed flush with the vial, then as air is puffed into the vial, it has nowhere to go, and will cause unwanted turbulence, disturbing successful placement of the insect. Hence either the tube is positioned at a distance above the vial, or the tube touches, requiring connector 164. The connector is preferably resistant to static charge.

Reference is now made to FIG. 19A which illustrates a screen 170 for use in manual tagging of the insects. If automatic tagging fails to generate a result, then the insect may be referred to a human operator who receives the images of the insect and manually identifies the species, gender etc. and tags accordingly. In an embodiment, the reference is made while the insect is still at the imaging station, and the software may allow the human operator to visually explore the insect by either rotating it or rotating the camera around it (or both).

The screen contains the current image 172 of the insect, and arrows below 174, 176, enable the human operator to move and take the next image according to an interval that may be preselected or which the operator may choose.

In a further embodiment, all the images are recorded, for example on a cloud service, which can enable operators to gain later access to each of the images, and either change the software decision as per the insect species, or to perform manual tagging as per the above. For example, humans may wish to review any decision whose confidence level is below a certain threshold or any species identification that seems unusual in some way.

FIG. 19B shows a screen 180 having multiple images 182.1 . . . 182.n in a sequence from an insect being rotated relative to the camera, and the operator may use the images to manually identify the species. A drop down list 184 of potential choices may be provided. If unsure or the operator requires assistance or a second opinion in making the identification, then an “ask the expert” or similarly named button 186 may be used to allow later identification. The expert is enabled to sort and filter the images to show only those marked with button 186. Radio button group 188 is provided for gender sorting.

In an embodiment, the identification of the insect may use images from other parts of the spectrum than light, or by sonic or ultrasonic sensors.

In embodiments, at the identification station, imaging may include hyper spectral imaging, reading reflections from a laser beam emitted towards the insect, use of a reagent which reacts to specific materials.

Once the insect or other material is identified, then it may be transferred into a corresponding vial.

In the case where a batch of insects is already known to be of the same species, say because of the type of trap, all that is needed is to place them into vials. The operator places the batch of insects into or onto the separator unit for separation into individuals and then the robotic tube takes each of the insects and transfers them into a corresponding vial using the pick and place suction unit described above. The method in such a case includes separation of insects, locating single insects on the separation surface, transfer of the separated insects into storage compartments by suction and then dropping or puffing them later into the storage area.

It is noted that the process may be used with different kinds of insects, such as mosquitoes, sand flies, fruit flies and in particular with a mixture of insects where the aim is say to study a particular ecosystem.

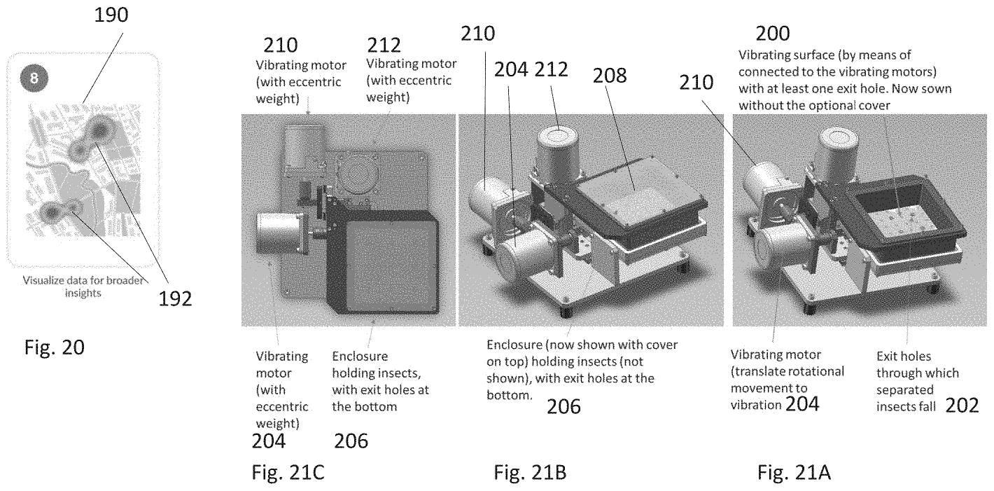

Reference is now made to FIG. 20, which illustrates the mapping stage. Insects may be gathered from multiple traps at different location and information may be required not just about the total number of insects but also about their distribution.

Mapping may be required but is not mandatory, and may happen in parallel to pooling so that different vials are used for collecting insects from different locations. Thus such information may be obtained as that the distribution of disease carrying organisms is limited to a certain part of the geographical distribution of the insects.

Once the mosquitoes from a given trap are identified and counted, then data of the location of the trap may be included with the insect count, because they are all from the same trap whose physical coordinates are known. Accordingly map 190 may be updated with information showing the number of insects in each trap. For example symbol 192 may visually indicate the number of insects of a given species over the location of the trap. Separate maps may be provided for different species, or different symbols may indicate different species, and the symbol may be overlaid with a color or with a percentage indicating the presence or proportion of disease carriers. Alternatively a report may provide numbers of insects with geographical location. The database may store the images of the insects alongside their locations.

The reports or maps may show timewise evolution of the insect population at a particular trap or over the geographical area. Timewise evolution may allow predictions to be made, say about spreading infestations.

The operator may enter the coordinates of the trap as each batch is emptied into the separator, or the separator may be present in situ and simply check its GPS coordinates each time it is filled, or the location may be entered in any other way that is convenient. Additional data about the environmental conditions at the trap may be entered, such as windspeed over the time the batch was obtained, altitude at the trap location and any other information that the user considers relevant.

Hence the operator may automatically update information on the geographical area represented on a map by introducing updated counting data. The data may automatically be streamed from the separating and identification machine as each trap is processed.

### Separation of the Insects:

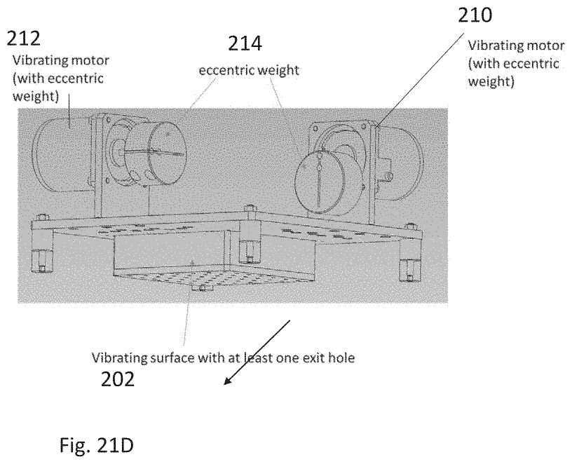

Reference is now made to FIGS. 21A . . . 21D, which show a further embodiment of the separator machine. The functionality of separating the insects was described hereinabove to vibrate the surface on which the insects are located. The vibration is timed with a rotation so that the machine rotates and then moves back and forth on a perpendicular axis causing vibrations. The vibrations may be on one axis but may alternatively be provided on two axes, to cause the insects to separate efficiently. One or more exit holes allow the insects to be ejected from the separation machine one by one. In an embodiment, the opening may have a phased shape at its exit hole, meaning the diameter at the outer side of the hole is larger than the diameter at the inner side to prevent clogging as discussed above.

In an embodiment, a typical entrance diameter may be 4mm with an exit diameter preferably of 6 mm. Other diameters are possible for different sizes of insects.

There may be one or more such exit holes on the vibrating surface. As described, the surface may be flat, or can be conical or rounded as in the case of the rotational storage compartment above.

The surface may itself include holes or the surface may be porous.

By means of vibrating the surface, the insects are separated, and by having exit holes on at least part of the vibrating surface, allows the separated insects to be ejected through the surface.

Adapters with different exit sizes may be attached to the exit holes and changed quickly, in order to use the same exit hole but adapt the machine for smaller insects.

When several different sizes of insects are found in the trap together, adapters may initially be applied to obtain the smaller insects first and then may be changed manually to provide a larger exit hole for larger insects. The process would typically be repeated as required until all or most of the insects are separated. Remaining insects may be identified manually.

As shown in FIGS. 21A . . . 21D, vibrating surface 200 of the separating machine according to the present embodiment has exit holes 202 through which the insects fall as the surface is vibrated by vibrating motor 204. The vibrating surface is the floor of enclosure 206 which has cover 208. Three vibrating motors 204, 210, 212 with eccentric weights 214 may be provided to apply vibrations in each axis.

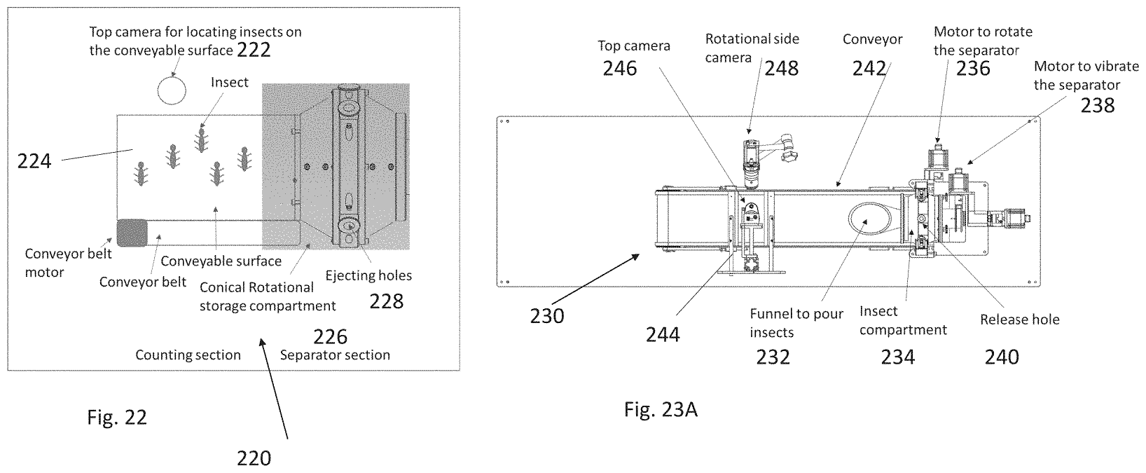

Referring now to FIG. 22, an embodiment is illustrated for circumstances in which the exact mosquito species is not required to be identified. In a variation only a single image, say a top view image, is sufficient to perform the required identification. For example it may only be required to determine the total number of mosquitoes, without identifying species or gender.

In apparatus 220, a sensor, such as top camera 222 is located to view the insects after ejection from the separator machine. The camera 222 may be located above a conveyable surface 224 which transfers the ejected insects away from separator 226, or the camera may be located below the ejecting holes 228 of separator 226, enabling the camera 222 to count falling insects on the fly. The camera may be replaced by a simple optic detector such as an IR detector, which only counts the number of falling objects regardless of whether they are mosquitoes or not. However a camera may provide images for computer vision software to verify what falls.

It is noted that the transfer of the individual elements towards an identification location may also be implemented by introducing an articulated robotic unit, or by an x-y (and potentially z) motorized axis moving a suction pipette, in either case having a suction pipette and being guided by a control system.

With regard to the main separator embodiment, with the rotating element, the expert may use multiple rotating elements over a single conveying system, and direct the operator to place batches of different size insects in each rotating drum, having different size openings for each.

In an embodiment, a separator unit may be installed inside a regular mosquito trap to be filled with insects and provide insects for imaging in real time.

In an embodiment, the pick and place device may be used directly on a batch of insects from a trap. The tube is sized to pick up the insects either one by one or in very small numbers and transfer them to the imaging location. If a large number of insects is inadvertently picked up by the pick and place device then the insects may be deposited and further attempts made to pick up individual insects.

Referring now to FIGS. 23A-23H, an embodiment is shown in which apparatus 230 include a funnel 232 for pouring of insects into insect compartment 234. The compartment is rotated by motor 236 and vibrated by motor 238 and insects are released one by one through release hole 240 onto conveyor 242 where they are conveyed to imaging area 244 where they are imaged by top camera 246 and rotational side camera 248 which moves around the insect as located by the top camera to obtain images at different angles. Light 249 illuminates the imaging area 244. A pick and place device consists of suction head 250 and motorized arm 252, and moves the insects from the imaging area 244 to the vials 254. Inlets 256 blow in air to free clogged holes by puffing the trapped insects back inside the separator machine. FIGS. 22D, 22G and 22H show a single motor 258 which both rotates and vibrates the storage compartment 234. Rotational side camera 248 is supported by holding structure 260, which may also support the top camera 246. Frame 262 may hold the separator, and a guide 264 may be provided to see that expelled insects get to conveyor 242. FIG. 23E shows motors 266, 268 and 270 for three axes of the robot arm 252.

The combination of the mechanical separators 230 and the conveyor 242 provides the ability to drop individual insects separately from each other, because as the rotational unit rotates, individual insects are ejected from the ejection holes. While they are being ejected, they have their own inertia because of the rotation, which causes each insect to land on the collecting surface, such as conveyor 242 at a different location and different time, supporting the physical separation between the insects as they travel towards the imaging station.

Reference has been made above to levels of confidence in an identification. When sending an image to a neural network model, there is always an answer. However, there may be no relevant body part in the image that is related to what we are looking for, so that the result provide from the model, represented as the probability for identifying a specific species in the image, should be very low. However, sometimes there are mistakes and the model may provide a high probability even for images where there is no relevant body part in the image.

In order to increase speed, calculation time and accuracy one embodiment may send for detection only images estimated to be from an angle relative to the insect to include relevant body parts with differentiating features. One way of doing this is using the top view camera to identify the posture of the insect. Accordingly, only the images at the angles corresponding to angles likely to show relevant information, are stored. One way to do this is to rotate the camera (or the insect) and take multiple images as usual but then only use specific images which correlate to particular angle with respect to the insect body axis.

In another embodiment, 3D imaging may be applied. In such a case the camera may not merely rotate around the insect in the same plane and at the same angle relative to the surface on which the insect is placed, but may also rotate and move up and down between planes, covering a 3D surface around the insect.

In one embodiment, a process to mechanically obtain the images is as follows:
Move conveyor 242 at a high speed A
Activate separator 230 by rotating and or vibrating the drum for X seconds. Once the separator is stopped, the conveyor 242 is moved at slow speed B towards imaging location 244. This is because separation may require a very high speed at the conveyor, so that it is advantageous to have breaks and work in a pulse mode. The mosquitoes are now separated along some 10 cm for example of the conveyor. The length of conveyor is now moved slowly, scanned, and after completion, the next cycle is entered starting with a high speed separation to produce another batch and create another area of 10 cm long of separated mosquitoes for scanning.

When the camera identifies insects in the top view, and sends images to identify the insect in the image, then the conveyor is stopped, the insect is located on the conveyor based on the coordinates found by the top camera and images are taken by the side camera to classify the insect.

Optionally a high resolution camera may take a top view above the indicated coordinate, and the side view camera may rotate in a circle whose center is the given coordinates, or a certain coordinate associated with the insect, say the insect head.

Images are taken at different angles between the camera and the insect (e.g. by rotating the camera relative to the insect). At each angle the camera may either take multiple images at different focus planes, in order to have focus at different body parts per that angle, or according to the top view camera, when calculating the coordinate, it may take into account the exact coordinate of the feature required to be in focus. When the camera takes an image the focus may thus be on the location that was calculated according to the posture. Changing the focus may be controlled using for example a liquid lens.

The conveyor is then restarted at the lower speed, until all insects on the conveyor are identified, and then a further batch of insects is separated and placed on the conveyor.

The insects are typically dead by the time they are imaged. Irrespective however of whether the insects are alive or dead, the conveyor may be cooled so that virus tests and the like are still viable.

The pulsing process described above provides the ability first to separate the insects on the conveyor in such that they are sufficiently separated, that is on average, not touching each other, and then to switch to imaging mode, where the separator stops, and the conveyor is moved slowly to identify insects on it, and then potentially completely stop to allow images from multiple positions around it. The conveyor may alternatively have a continuous slow motion and multiple camera mounts around positions above the conveyor, so that multiple insects may be identified in parallel.

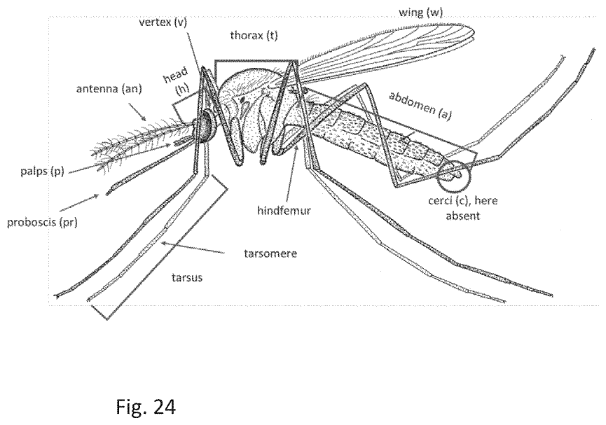

Reference is now made to FIG. 24, which is a generic figure showing features of interest on a mosquito that can become relevant in finding the species or gender of the mosquito. These include the head, antennae, palps, proboscis, vertex, thorax, abdomen, wings and their patterns, legs and leg parts, and cerci.

Reference is now made to table 1, which illustrates a decision tree for finding out the species of mosquito. The neural network model receives images where the body parts are supposed to be located, and then applies such a decision scheme to decide which species it is. Typically the decision tree tells the network which body parts to look for. It either recognizes or does not recognizes the body parts in the image and uses these recognitions to navigate through the decision tree to a decision.

Other decision schemes are possible. Also, it is possible to train a neural network to identify the species according to a set of images it received per each body part.

TABLE 1
Decision tree for Mosquito species

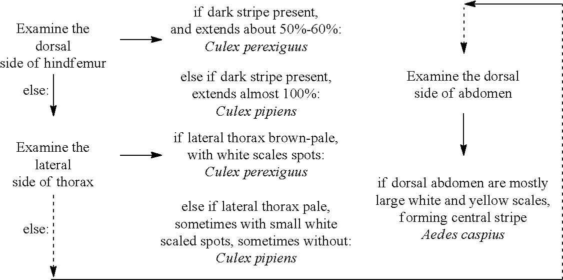

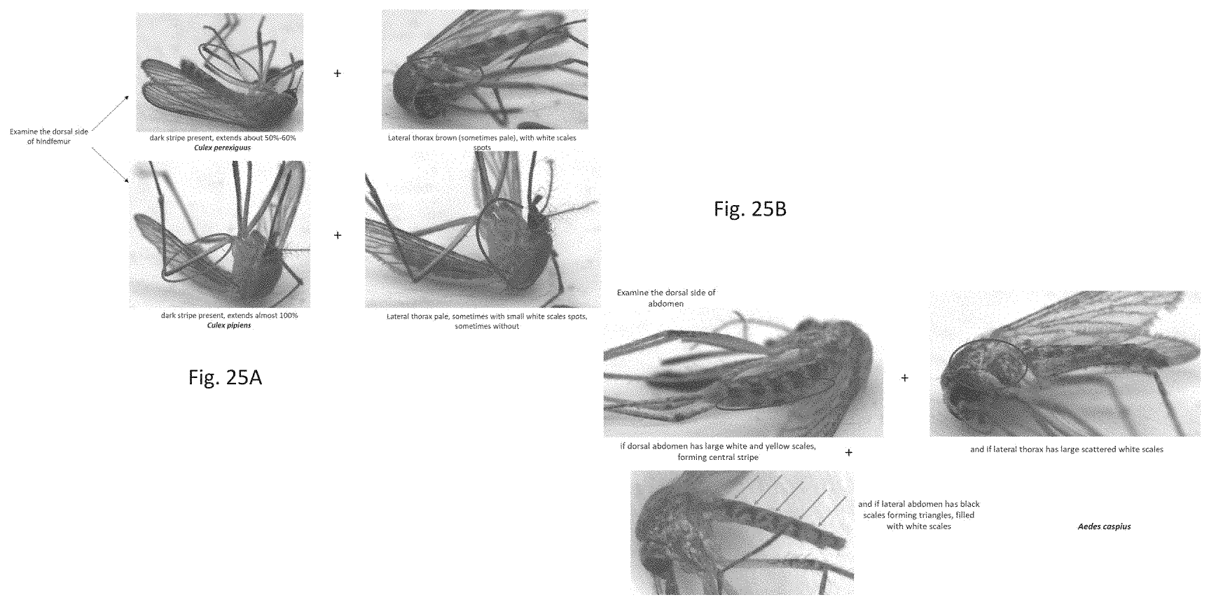

FIG. 25A shows an example of how the decision tree may be used to identify a sample of Culex Pipiens.

In the dorsal view of the hind femur, a dark stripe present which extends over almost 100% of the segment length.

The lateral thorax is pale, and is usually without small white scales spots, but sometimes is with such spots.

FIG. 25B shows an example of how the decision tree may be used to identify Aedes Caspius.

There are some 3,500 different species of mosquito around the world, yet in each specific location (a country, country region, a city, a neighborhood) there is a subset of species which are commonly found, hence, during system calibration, and as an important pre-stage process, the set of species the model needs to compare the pictures with is entered. Thus the system may be calibrated to the species expected in the trap or batch.

In one embodiment, the separator machine may be dispensed with, and individual insects may be placed by a human operator on the imaging station for imaging in the way described above. Such a process is particularly advantageous for initial training of the neural network.

A variation of the processes discussed hereinabove involves fusion of the classification of body parts and identification using emitted fluorescent light to obtain a classification of species.

A light source is provided which emits a wavelength that can excite a fluorescence response from the insect at an imaging station. Specific body parts of the insects are excited and emit fluorescence light. In the process, Imaging is initially carried out to obtain a top view using visible light to determine if there is an insect in the image. If there is an insect, the top view image may be used to identify at least one body part, say the head, and give its coordinates

Imaging is then carried out, again using the top view with light of the appropriate excitation wavelength to excite the body part to emit light at its own fluorescing wavelength. The fluorescing wavelength may be detected at the camera, say with a suitable filter.

The coordinate of the fluorescing light identified in the image is compared with the location of the body part identified in the top camera view, to correlate and identify that a specific body part was emitting the light. In some cases, classification of the insects ends if the insect emits a specific wavelength but in other cases it may be required to count how many insects emit a specific wavelength from a specific body part. Thus for example ten male mosquitoes of aedes albopictus with red eyes may be identified, and 3 female mosquitoes of the type aedes albopictus with green fluorescence from the head may be identified.

The method may optionally use a side camera to provide support in identifying the insect species using the methods described above for using a side camera, and indeed classification of the body part emitting the light may be carried out using the side camera.

Another option is to completely separate between an identification made using visible light and an identification using fluorescence. Such a separation may optimize the process, thus the insect may be imaged as above using a rotational camera, and then may be imaged again using the excitation wavelength for florescence excitation.

The process may use the methods described above to separate the insects and drop them on a conveyor, taking first a top view image then a side view image and classify using normal light. The conveyor then moves to a second imaging fluorescence station. At the second imaging station the insect is imaged to locate specific body parts and their coordinates such as the head using visible light. Then the excitation frequency is used and the fluorescence wavelength is detected by the camera. The coordinates of the detected fluorescence are compared with the coordinates of the body part expected to have produced the fluorescence.

Different body parts may emit different wavelengths, and then classification may classify the insects both by their species and also which wavelength they emitted.

Optional picking of the insects and placement into vials or storage containers, may also be according to the fluorescence identification together with the species, for example aedes aegypti with a head that fluoresces orange may be placed in one vial, and aedes aegypti with a green fluorescing abdomen may be placed in a different vial.

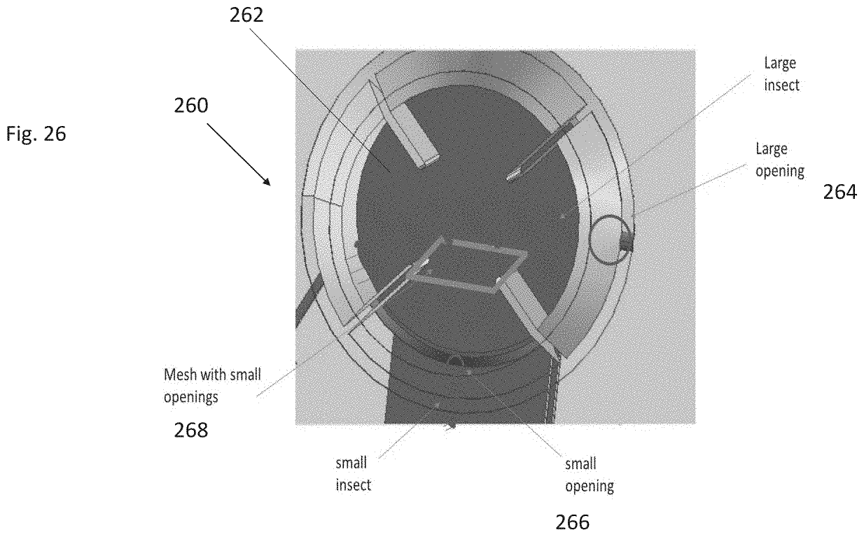

Reference is now made to FIG. 26, which is a simplified diagram showing a device for separating insects of different sizes at the same time. The device 260 is a variation of separator machine 11, in which the exit holes are in floor 262 and are of different sizes. Exit holes 264 and 266 are of different sizes and a mesh 268 moves between the different holes. The floor is vibrated and the mesh moves between the different sized holes, so that at part of the time, the larger hole is in operation and at other times the smaller hole is in operation. More than two different sized holes may be used.

It is expected that during the life of a patent maturing from this application many relevant imaging and neural network technologies will be developed and the scopes of these and other terms herein are intended to include all such new technologies a priori.
The terms “comprises”, “comprising”, “includes”, “including”, “having” and their conjugates mean “including but not limited to”.
The term “consisting of” means “including and limited to”.
As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of separating a batch of insects from a trap into individual insects comprising:
pouring said batch of insects into a container having at least one hole, the hole being sized for a single insect;
moving the container with a shaking motion, thereby to shake the insects within so that individual insects are caused to exit via said hole onto a collecting surface, thereby providing separated insects onto said collecting surface.

2. The method of claim 1, wherein said container comprises a floor, said motion comprises vibration, and said at least one hole is in said floor.

3. The method of claim 1, wherein said container comprises a circumference, said at least one hole is in said circumference and said motion comprises rotation.

4. The method of claim 3, wherein said motion further comprises vibration.

5. The method of claim 4, wherein said rotation and said vibration are alternated in a cycle.

6. The method of claim 3, wherein said container comprises an upper cone and a lower cone, said cones meeting at a common base, said base providing a maximal circumference and said at least one hole being at said maximal circumference.

7. The method of claim 1, comprising pouring a batch of insects into said container via a funnel.

8. The method of claim 1, wherein said collecting surface is a moving surface.

9. Apparatus for separating insects from a batch of insects into individuals, the apparatus comprising a container for said batch of insects, the container being motorized to provide motion to the container to shake said insects in a shaking motion, and having at least one hole, the hole sized for an individual insect thereby to enable an individual insect from said batch to be pushed out of said hole when nudged against said hole by said shaking motion.

10. Apparatus according to claim 9, wherein the container has a floor, said at least one hole is in said floor and said shaking motion comprises vibrating motion.

11. Apparatus according to claim 9, wherein the container has a circumference, said motion comprises rotation in an axis perpendicular to said circumference and said at least one hole is in said circumference.

12. Apparatus according to claim 11, wherein said motion further comprises vibration in at least one axis.

13. Apparatus according to claim 12, wherein said motion comprises vibration in three axes.

14. Apparatus according to claim 9, wherein said at least one hole comprises an inner side towards an interior of said container and an outer side towards an exterior of said container, and a diameter which is smaller at said inner side than at said outer side.

15. Apparatus according to claim 9, wherein said container comprises an upper cone and a lower cone, said cones meeting at a common base, said base providing a maximal circumference and said at least one hole being at said maximal circumference.

16. Apparatus according to claim 9, having a guide for guiding exiting insects from said at least one hole to a collecting surface.

17. Apparatus according to claim 9, comprising at least one member of the group consisting of:
a funnel for pouring said batch of insects from a trap into said container; and
a motor with an eccentric weight to provide vibrations.

18. (canceled)

19. A method of picking an insect on a first surface and placing said insect, the method comprising:
Imaging said collecting surface from above;
From said imaging determining the presence of said insect on said surface for picking;
From said imaging determining a current location of said insect on said surface as a picking location;
Using a robot arm, moving a picking tool to a positon above said picking location;
Lowering said picking tool to said picking location;
Operating suction to pick said insect into said picking tool from said picking location;
Using said robot arm to move said picking tool with said insect to a position above a depositing location; and
Removing said suction to deposit said insect, wherein one of said picking location and said depositing location is an identification location for imaging said insect for identification, wherein said picking tool comprises a porous surface in a tube leading to a vacuum source, said insect being held at said porous surface by said suction.

20. The method of claim 19, wherein said identification location is said picking location and an identification made at said identification location defines said depositing location.

21. The method of claim 19, comprising switching from said suction to blowing at said depositing location to deposit said insect.

22. (canceled)

23. The method of claim 19, wherein said picking tool has a central air duct and a peripheral air duct, said suction being applied via said central air duct and said blowing being provided by both said central air duct and said peripheral air duct.

24. A picking tool for insects comprising a hollow tube having a first end and a second end, the tube being connected to an air pressure source at said first end and having a porous surface proximal to said second end, the tool further having a robot arm for positioning the tool in three dimensions, the tool being configured to work with an imaging system to position itself above coordinates supplied by said imaging system as the position of an insect on a surface, the tool being configured to lower itself over said coordinates and to apply suction to suck said insect against said porous surface thereby to pick said insect.

25. The picking tool of claim 24, wherein said net is distanced from said second end by the thickness of an insect.

26. The picking tool of claim 24, having a central air duct and a peripheral air duct, said suction being applied through said central air duct, thereby to positon said picked insect centrally on said net.

27. The picking tool of claim 26, configured to switch off said suction when reaching a destination, thereby to deposit said insect at a placing location, or configured to switch off said suction when reaching a destination, and to replace said suction with blowing, said blowing being applied via said central air duct and said peripheral air duct, thereby to deposit said insect at said placing location.

28-50. (canceled)
